(19) United States
(12) Patent Application Publication
(10) Pub. No.: US 2010/0247659 A1
(43) Pub. Date: Sep. 30, 2010

(54) PHENYLPHTHALIMIDE ANALOGS FOR TREATING DIABETIC MACULAR EDEMA

(75) Inventors: Dumitru Ionescu, Ann Arbor, MI (US); CherylAnn Love, Warren, MI (US); Danyang Chen, Oklahoma City, OK (US); Ronald Andrew Wassel, Oklahoma City, OK (US)

Correspondence Address: FOLEY HOAG, LLP;PATENT GROUP, WORLD TRADE CENTER WEST, 155 SEAPORT BLVD, BOSTON, MA, 02110, US

(73) Assignee: Charlesson, LLC, Oklahoma City, OK (US)

(21) Appl. No.: 12/509,369

(22) Filed: Jul. 24, 2009

Related U.S. Application Data
(63) Continuation-in-part of application No. 11/760,192, filed on Jun. 8, 2007, now abandoned.

(30) Foreign Application Priority Data
Jun. 6, 2008 (US) PCT/US08/66064

(51) Int. Cl. A61K 31/4035; A61K 9/50; C07D 209/48; A61P 27/02

(52) U.S. Cl. 424/490; 514/417; 548/476

(57) ABSTRACT

Novel methods are provided to prevent blindness associated with diabetic macular edema by administration of a phenylphthalimide analog. Additionally, sustainability of the effect of administration of the phenylphthalimide analog is improved via encapsulation with poly(lactic-co-glycolic acid) nanoparticles. Finally, a novel method for synthesizing (2,6-diisopropylphenyl)-5-amino-1H-isoindole-1,3-dione is disclosed.

### CROSS-REFERENCE TO RELATED APPLICATIONS

This application is a continuation-in-part of, incorporates by reference, and claims the priority of U.S. Utility patent application Ser. No. 11/760,192, filed Jun. 8, 2007.

### FEDERALLY SPONSORED RESEARCH

This work is supported by the National Eye Institute grants (NIH EY016627-01, EY017229-02, and EY017229-03).

### BACKGROUND

It has been found that thalidomide, a potent anti-angiogenic and phenylphthalimide approved for the treatment of multiple myeloma and erythema nodosum leprosum, blocked the increase of VEGF in ocular fluid and inhibited the thickening of retinal capillary basement membrane in STZ-diabetic rats, thus representing a potential therapeutic drug for the treatment of the effects of diabetic retinopathy, including diabetic macular edema.

Additionally, a novel method of synthesizing (2,6-diisopropylphenyl)-5-amino-1H-isoindole-1,3-dione (Compound 4, CLT-003) is disclosed.

### SUMMARY

Novel methods are provided to prevent blindness associated with diabetic macular edema by administration of a phenylphthalimide analog. Additionally, sustainability of the effect of administration of the phenylphthalimide analog is improved via encapsulation with poly(lactic-co-glycolic acid) nanoparticles. Finally, a novel method for synthesizing (2,6-diisopropylphenyl)-5-amino-1H-isoindole-1,3-dione is disclosed.

According to a feature of the present disclosure, a method is disclosed comprising administering to a subject having retinal edema a pharmaceutical composition having the chemical structure:

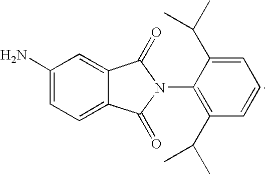

According to a feature of the present disclosure, a method is disclosed comprising providing a pharmaceutical composition to a patient experiencing retinal edema, the pharmaceutical composition comprising an agent having the chemical structure:

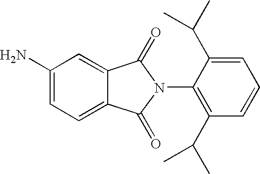

encapsulated in a poly(lactic-co-glycolic acid) nanoparticle.

According to a feature of the present disclosure, a method is disclosed comprising sustaining the efficacy of a pharmaceutical composition comprising a compound having the structure:

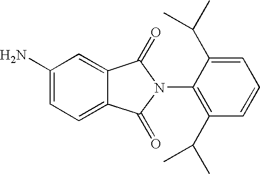

by encapsulating the compound in a poly(lactic-co-glycolic acid) nanoparticle. Subsequent delivery of the encapsulated capsule causes the therapeutic effect of the compound for the treatment of diabetic macular edema to be extended.

According to a feature of the present disclosure, a method is disclosed comprising synthesizing a composition of the formula:

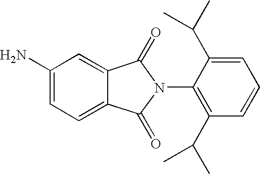

by coupling 4-nitrophthalic anhydride and 2,6-diisopropyl aniline by refluxing in acetic acid to produce (2,6-diisopropylphenyl)-5-nitro-1H-isoindole-1,3-dione; and hydrogenating the (2,6-diisopropylphenyl)-5-nitro-1H-isoindole-1,3-dione by reacting the (2,6-diisopropylphenyl)-5-nitro-1H-isoindole-1,3-dione via a catalytic hydrogenation or transfer hydrogenation reaction.

### DRAWINGS

The above-mentioned features and objects of the present disclosure will become more apparent with reference to the following description taken in conjunction with the accompanying drawings wherein like reference numerals denote like elements and in which:

FIG. 1 is a table showing the effect of thalidomide and its phenylphthalimide analogs on cell proliferation;

FIG. 2 is a collection of diagrams showing the inhibition of endothelial cell (HUVEC) migration by Compound 1, Compound 4, and thalidomide;

FIG. 3 is a collection of images showing the effect of Compound 4 on tube formation;

FIG. 4 is a collection of images showing the effect of Compound 4 on blood vessel formation in CAM assay;

FIG. 5 is a collection of images showing the effect of phenylphthalimide analogs on HIF-1α expression;

FIG. 6 is a collection of images showing that Compound 4 down-regulated the expression of VEGF;

FIG. 7 is a collection of graphs showing the effect of thalidomide, Compounds 1, 2, and 4 on retinal vascular leakage in OIR rats;

FIG. 8 is a collection of graphs showing the effect of thalidomide, Compounds 1, 2 and 4 on retinal vascular leakage in STZ-diabetic rats;

FIG. 9 is a collection of images showing retinal angiographs of OIR rats with a single intravitreal injection of thalidomide and Compounds 1 and 4;

FIG. 10 is graph showing the rat strain difference in vascular permeability in the OIR model;

FIG. 11 is a collection of graph showing the strain difference in vascular permeability in STZ-diabetic model;

FIG. 12 is a bar graph showing VEGF levels in OIR BN and SD rats;

FIG. 13 is an image showing retinal VEGF levels in BN and SD rats with STZ-diabetes;

FIG. 14 is a table showing the effect of Compound 4 on blood vessel formation in CAM assay;

FIG. 15 is a table showing the effect of Compound 4 on the A wave and B wave of eyes in rats;

FIG. 16 is a diagram showing route of synthesis for Compound 4;

FIG. 17 is a collection of diagrams showing the chemical structures of thalidomide and the phenylphthalimide analogs;

FIG. 18 is a collection of images showing the functional and morphological analysis of the retina treated by Compound 4 in rats;

FIG. 19 is a collection of images showing the effect of Compound 4 on HIF-1α activation;

FIG. 20 are graphs showing the effect of Compound 4 on the expression of VEGF and soluble ICAM-1 in ARPE-19 cells;

FIG. 21 are graphs showing the effect of Compound 4 on the expression of VEGF and ICAM-1 in the retina of OIR rats;

FIG. 22 are graphs showing the effect of Compound 4 retinal vascular leakage in OIR and STZ-diabetic rats;

FIG. 23 are graphs showing the effect of Compound 4 on retinal vascular leakage in STZ-diabetes rats after oral administration;

FIG. 24 is a scanning electron micrograph of Compound 4 PLGA nanoparticles.

FIG. 25 are graphs showing the effect of Compound 4 nanoparticles on retinal vascular leakage in STZ-diabetes rats; and

FIG. 26 are graphs showing the inhibitory activity of nanoparticles and unincorporated Compound 4 on BRCEC growth.

### DETAILED DESCRIPTION

In the following detailed description of embodiments of the present disclosure, reference is made to the accompanying drawings in which like references indicate similar elements, and in which is shown by way of illustration specific embodiments in which the present disclosure may be practiced. These embodiments are described in sufficient detail to enable those skilled in the art to practice the present disclosure, and it is to be understood that other embodiments may be utilized and that logical, mechanical, electrical, functional, and other changes may be made without departing from the scope of the present disclosure. The following detailed description is, therefore, not to be taken in a limiting sense, and the scope of the present disclosure is defined only by the appended claims. As used in the present disclosure, the term “or” shall be understood to be defined as a logical disjunction and shall not indicate an exclusive disjunction unless expressly indicated as such or notated as “xor.”

As used above and elsewhere herein the following terms, acronyms, or abbreviations have the following meanings: AMD is an acronym for age-related macular degeneration; bFGF is an acronym for basic fibroblast growth factor; BN is an acronym for Brown-Norway; BSA is an acronym for bovine serum albumin; CAM is an acronym for chorioallantoic membrane; Compound 4 (CLT-003) is an abbreviation for (2,6-diisopropylphenyl)-5-amino-1H-isoindole-1,3-dione); Compound 1 is an abbreviation for (2,6-diisopropylphenyl)-5-hydroxy-1H-isoindole-1,3-dione; Compound 2 is an abbreviation for (2,6-diisopropylphenyl)-1H-isoindole-1,3-dione; DME is an acronym for diabetic macular edema; DR is an acronym for diabetic retinopathy; DMSO is an acronym for dimethyl sulfoxide; EPO is an acronym for erythropoietin; ERG is an acronym for electroretinogram; HIF-1 is an acronym for hypoxia induced factor-1; HUVEC is an acronym for human umbilical vein endothelial cells; IGF-1 is an acronym for insulin-like growth factor; NV is an acronym for neovascularization; OIR is an acronym for oxygen-induced retinopathy; PEG is an acronym for polyethylene-glycol; PET is an acronym for polyethylene terepthlate; ROP is an acronym for retinopathy of prematurity; RPE is an acronym for retinal pigment epithelial; SD is an acronym for sprague-Dawley; STZ is an acronym for streptozotocin; VEGF is an acronym for vascular endothelial growth factor; and VEGFR is an acronym for vascular endothelial growth factor receptor

The term “angiogenesis” is the generation of new blood vessels into a tissue or organ.

The term “retinal vascular leakage” is defined as the increase of retinal vascular permeability.

The phrase “therapeutically effective amount” is recognized in the art when used in reference to an amount of the therapeutic agent that produces some desired effect at a reasonable benefit/risk ratio applicable to any medical treatment. The effective amount may vary depending on such factors as the disease or condition being treated, the particular targeted constructs being administered, the size of the subject, or the severity of the disease or condition. One of ordinary skill in the art may empirically determine the effective amount of a particular compound without necessitating undue experimentation.

The term “treatment” is recognized in the art and includes inhibiting or impeding the progress of a disease, disorder or condition and relieving or regressing a disease, disorder, or condition. Treatment of a disease or condition includes ameliorating at least one symptom of the particular disease or condition, even if the underlying pathophysiology is not affected, such as treating the pain of a subject by administration of an analgesic agent even though such agent does not treat the cause of the pain.

Diabetic retinopathy (DR) is a common microvascular complication of diabetes mellitus and remains a leading cause of severe vision loss and blindness. Retinal vascular leakage caused by the breakdown of blood-retina barrier is an early and common pathological alteration in DR. It is confirmed that the increase of retinal vascular leakage precedes the appearance of clinical retinopathy at early stages of DR. Retinal vascular leakage often leads to diabetic macular edema (diabetic macular edema), which is the single greatest cause of vision loss in diabetes.

Diabetic macular edema is characterized by the accumulation of extracellular fluid in Henle's layer and the inner nuclear layer of the retina and can develop at any time in the progression of DR. Current interventions for DR, including laser photocoagulation and vitrectomy, impede visual loss but usually do not improve visual acuity for most patients with DR. Due to the absence of an early and adequate treatment, DR remains to be a major cause for visual loss and blindness in industrialized countries. Thus, there is a need to develop new therapies for DR/diabetic macular edema.

Use of a small molecule compound, Compound 4 (CLT-003) has shown to be effective in the treatment of diabetic macular edema. Research data demonstrated that Compound 4 possesses both anti-angiogenic and anti-inflammatory activities. Compound 4 attenuated the activation of hypoxia-inducible factor-1alpha (HIF-1α) and down-regulated the expression of vascular endothelial growth factor (VEGF) and intercellular adhesion molecule-1 (ICAM-1) in human retinal pigment epithelial cells and the retina of oxygen induced retinopathy (OIR) rats. Compound 4 also significantly reduced retinal vascular leakage in OIR and streptozotocin (STZ)-induced diabetes rats. Additionally, a single intravitreal injection of compound 4 poly(lactic-co-glycolic acid) (PLGA) nanoparticles (NPs) has displayed prolonged efficacy on reduction of retinal vascular leakage for at least six weeks in STZ-diabetes rats. These findings suggest that this single molecule has multiple beneficial effects and has great potential to become a new drug for the treatment of macular edema, in particular diabetic macular edema.

The compounds of the present disclosure that have one or more asymmetric carbon atoms may exist as optically pure enantiomers, optically pure diastereomers, mixtures of enantiomers, mixtures of diastereomers, or racemic mixtures of the stereoisomers. The present disclosure includes within its scope all such isomers and mixtures thereof.

The present disclosure relates to novel compounds of phenylphthalimide analogs that have anti-angiogenic and anti-vascular permeability activity. More particularly, the disclosure is directed to a series of phenylphthalimide analogs wherein the piperidine-2,6-dione moiety has been replaced with 2,6-diisopropylaniline as shown below:

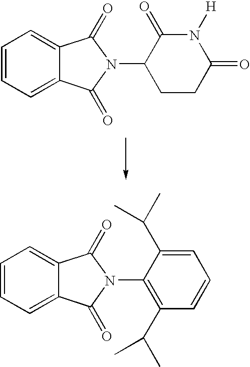

In accordance with one aspect of the present disclosure, a novel compound is provided having a general structure:

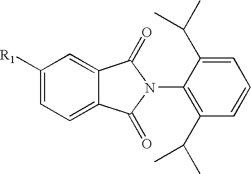

wherein R₁ is selected from a group comprising of hydroxy, hydrogen, and amino.

As used above and elsewhere herein, Compound 1 is the embodiment of the compound wherein R₁ is a hydroxy, Compound 2 is the embodiment of the compound wherein R₁ is a hydrogen, and Compound 4 is the embodiment of the compound wherein R₁ is an amino. The various embodiments are shown below:

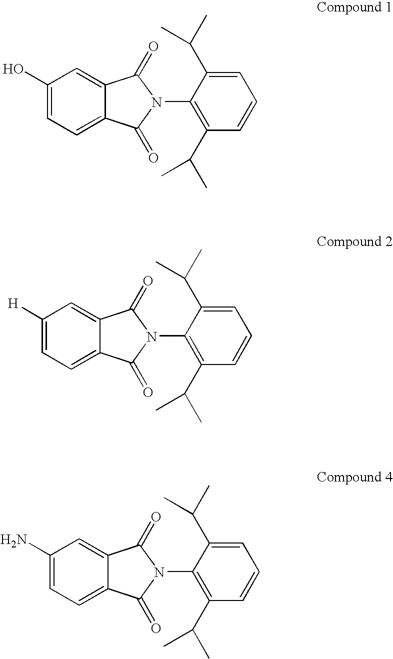

In accordance with a further aspect of the present disclosure, an anti-angiogenic and anti-vascular permeability compound is provided having a general structure:

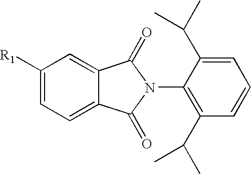

wherein R₁ is selected from a group comprising of hydroxy, hydrogen, and amino.

According to embodiments useful for the treatment of diabetic macular edema, the compound has the structure:

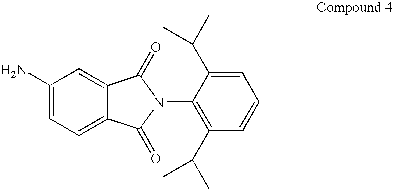

In accordance with another aspect of the present disclosure, a method is provided for treating vascular abnormalities in a patient. More particularly, one embodiment of the disclosure is directed to treating neovascularization and/or vascular leakage. The method comprises administering to the patient a therapeutically effective amount of a composition comprising a compound of the general structure:

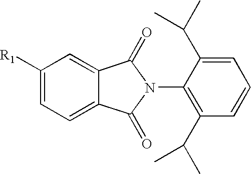

wherein R₁ is selected from a group comprising of hydroxy, hydrogen, and amino.

In particular, though not exclusively, the treatment is targeted towards retinal vascular abnormalities, including diabetic retinopathy, diabetic macular edema, age-related macular degeneration, sickle cell retinopathy, retinal vein occlusion, retinopathy of prematurity, and other forms of retinopathy and diseases resulting from retinal neovascularization or retinal vascular leakage. In one embodiment, the treatment includes suppressing VEGF as well as HIF-1α, a major transcription factor up-regulating VEGF in diabetic retina. Additionally, the phenylphthalimide analogs may also be used as sodium channel Mockers, calcium channel Mockers, contraceptives, anti-inflammatory agents and anti-cancer agents.

In another aspect of the present disclosure, a phenylphthalimide analog is synthesized by substituting the glutaramide ring with an aromatic group. In one embodiment, Compound 4 is synthesized by using the reactants 4-nitrophthalic anhydride and 2,6-diisopropylaniline to produce (2,6-diisopropylphenyl)-5-nitro-1H-isoindole-1,3-dione, which is further processed to form Compound 4. According to an embodiment, 4-nitrophthalic anhydride and 2,6-diisopropylaniline is refluxed with AcOH for 3 hrs to produce (2,6-diisopropylphenyl)-5-nitro-1H-isoindole-1,3-dione. (2,6-diisopropylphenyl)-5-nitro-1H-isoindole-1,3-dione is further refluxed with H₂, Pd/C, and ethanol for 2 hrs to form Compound 4.

Structure-activity-relationship studies showed that substituting the glutaramide ring of thalidomide with an aromatic group leads to active analogs. Specifically, replacing the glutaramide ring with 2,6-diisopropylaniline yielded more active anti-angiogenic and anti-vascular permeability analogs—Compounds 1, 2, and 4.

In vitro screening using endothelial cell proliferation assay has demonstrated that three of the compounds, Compounds 1, 2, and 4 have potent anti-proliferative activities, as they selectively inhibited HUVEC and BRCEC growth with an IC₅₀ of <3.3 μM, which was substantially lower than that of thalidomide, and existing phenylphthalimide analogs Actimid and Revimid (IC₅₀>100 μM). In addition, the phenylphthalimide analogs did not inhibit the growth of non-endothelial cells, such as pericytes (IC_{50>32} μM), suggesting that the inhibition to endothelial cell growth is cell type-specific rather than a result of non-specific cytotoxicities. One of the phenylphthalimide analogs, Compound 4 displayed potent effects on growth of HUVECs and BRCECs (IC_{50<3.3} μM), the migration of HUVECs (IC₅₀ of <1 μM), the tube formation of HUVECs, and vascular formation in the CAM assay (ED₅₀=6.5 μg/embryo). The anti-angiogenic effect of Compound 4 was also demonstrated in the OIR model, a commonly accepted model for retinal NV and for proliferative diabetic retinopathy.

The effects of thalidomide and novel phenylphthalimide analogs on retinal vascular leakage and NV have been compared in OIR and STZ-diabetic rats. The STZ-diabetic rats are a widely used model of experimental diabetes since the diabetic rats develop background diabetic retinopathy including vascular leakage. The OIR model is also shown to develop abnormal vascular leakage in the retina. The experimental results showed that the novel phenylphthalimide analogs had significantly more potent effects on retinal vascular leakage than thalidomide in both animal models.

Compound 4 and thalidomide, at a single dose of 1.0 μg/eye, reduced retinal vascular leakage by 40% and 18% respectively when compared with vehicle control in OIR rats. In STZ-induced diabetic rats, Compound 4, thalidomide, and Compound 1 at a single dose of 1.0 μg/eye reduced retinal vascular leakage by 100%, 77% and 61%, respectively, when compared with vehicle control. Twenty-four and 48 hours after a single administration, Compound 4 completely blocked retinal vascular leakage induced by diabetes. Compound 4 reduced retinal vascular leakage in a dose-dependent manner. These results indicate that Compound 4 has a potent effect on reduction retinal vascular leakage not only in the OIR model but also in the STZ-diabetes model, compared to thalidomide.

The regulatory effect of Compound 4 on VEGF expression in the retina of OIR rats has also been investigated with results showing that Compound 4 down-regulates the expression of VEGF. This suggests that Compound 4 targets signaling from VEGF.

The experiments have shown that Compound 4 inhibits cell proliferation in HUVEC and BRCEC, but not in non-endothelial cells, suggesting that its effect is endothelial cell-specific. Compound 4 was chosen to assess the potential ocular toxicity in rats. ERG recording and histopothological examination both demonstrated that Compound 4, at a single high dose, does not result in detectable changes in the ocular function and morphology in rats. The results imply that Compound 4 lacks significant toxicities at doses required for its anti-angiogenic and anti-vascular permeability activities.

These experiments have shown that a low dose of Compound 4 can inhibit NV and anti-vascular permeability. The more potent antiangiogenic and anti-vascular permeability effects of Compound 4 suggest that low doses of the compound are sufficient to achieve inhibition of NV and anti-vascular permeability, and are therefore less likely to cause side effects.

Proteinuria in diabetic nephropathy is another type of vascular leakage. Compound 4 may also be applied to treat proteinuria due to its effect in reducing leakage of macromolecules out of blood vessels. Vascular leakage is an essential step in tumor metastasis. Blockage of vascular leakage of tumor vessels is also expected to have beneficial effect in solid tumor treatment.

According to embodiments, compound 4 attenuated HIF-1α activation and down-regulated the expression of VEGF and ICAM-1. Moreover, a single intravitreal injection and oral administration of compound 4 reduced retinal vascular leakage, suggesting that compound 4 may have therapeutic potential to treat diabetic macular edema. Finally, the nanoparticle-mediated Compound 4 delivery produced sustained reduction of retinal vascular leakage in diabetes rats.

### Pharmaceutical or Nutraceutical Compositions

According to another aspect, the compounds of this disclosure can be included in a pharmaceutical or nutraceutical composition together with additional active agents, carriers, vehicles, excipients, or auxiliary agents identifiable by a person skilled in the art upon reading of the present disclosure.

The pharmaceutical or nutraceutical compositions preferably comprise at least one pharmaceutically acceptable carrier. In such pharmaceutical compositions, one or more of the compounds of this disclosure forms the “active compound,” also referred to as the “active agent.” As used herein the language “pharmaceutically acceptable carrier” includes solvents, dispersion media, coatings, antibacterial and antifungal agents, isotonic and absorption delaying agents, and the like, compatible with pharmaceutical administration. Supplementary active compounds can also be incorporated into the compositions. A pharmaceutical composition is formulated to be compatible with its intended route of administration. Examples of routes of administration include parenteral, e.g., intravenous, intradermal, subcutaneous, oral (e.g., inhalation), transdermal (topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol, or other synthetic solvents; antibacterial agents such as benzyl alcohol or methyl parabens; antioxidants such as ascorbic acid or sodium bisulfite, chelating agents such as ethylenediaminetetraacetic acid; buffers such as acetates, citrates, or phosphates and agents for the adjustment of tonicity such as sodium chloride or dextrose. The pharmaceutical compositions can also be administered through ocular routes including: intravitreal, subconjunctival, intracameral, episcreal, retrobulbar, sub-tenon, or subretinal injections, or via topical eye drop. pH can be adjusted with acids or bases, such as hydrochloric acid or sodium hydroxide. The parenteral preparation can be enclosed in ampoules, disposable syringes, or multiple dose vials made of glass or plastic.

Subject as used herein refers to humans and non-human primates (e.g., guerilla, macaque, marmoset), livestock animals (e.g., sheep, cow, horse, donkey, and pig), companion animals (e.g., dog, cat), laboratory test animals (e.g., mouse, rabbit, rat, guinea pig, hamster), captive wild animals (e.g., fox, deer), and any other organisms who can benefit from the agents of the present disclosure. There is no limitation on the type of animal that could benefit from the presently described agents. A subject regardless of whether it is a human or non-human organism may be referred to as a patient, individual, animal, host, or recipient.

Pharmaceutical compositions suitable for an injectable use include sterile aqueous solutions or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In all cases, the composition should be sterile and should be fluid to the extent that easy syringability exists. It should be stable under the conditions of manufacture and storage and be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, or sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle which contains a basic dispersion medium and the required other ingredients from those enumerated above. In the case of sterile powders for the preparation of sterile injectable solutions, methods of preparation include vacuum drying and freeze-drying, which yields a powder of the active ingredient plus any additional desired ingredient from a previously sterile-filtered solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier for use as a mouthwash. Pharmaceutically compatible binding agents, or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser which contains a suitable propellant, e.g., a gas such as carbon dioxide, or a nebulizer.

Systemic administration can also be transmucosal or transdermal. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration may be accomplished through the use of nasal sprays or suppositories. For transdermal administration, the active compounds are formulated into ointments, salves, gels, or creams as generally known in the art. The compounds can also be prepared in the form of suppositories (e.g., with conventional suppository bases such as cocoa butter and other glycerides) or retention enemas for rectal delivery.

In addition to the other forms of delivery, the compounds are deliverable via eye drop or intraocular injection. With respect to eye drops, the compositions of the present disclosure optionally comprise one or more excipients intended for topical application to the eye or nose. Excipients commonly used in pharmaceutical compositions intended for topical application to the eyes, such as solutions or sprays, include, but are not limited to, tonicity agents, preservatives, chelating agents, buffering agents, surfactants and antioxidants. Suitable tonicity-adjusting agents include mannitol, sodium chloride, glycerin, sorbitol and the like. Suitable preservatives include p-hydroxybenzoic acid ester, benzalkonium chloride, benzododecinium bromide, polyquaternium-1 and the like. Suitable chelating agents include sodium edetate and the like. Suitable buffering agents include phosphates, borates, citrates, acetates and the like. Suitable surfactants include ionic and nonionic surfactants, though nonionic surfactants are preferred, such as polysorbates, polyethoxylated castor oil derivatives and oxyethylated tertiary octylphenol formaldehyde polymer (tyloxapol). Suitable antioxidants include sulfites, ascorbates, BHA and BHT. The compositions of the present disclosure optionally comprise an additional active agent. With the exception of the optional preservative ingredient (e.g., polyquaternium-1), the compositions of the present disclosure preferably do not contain any polymeric ingredient other than polyvinylpyrrolidone or polystyrene sulfonic acid.

When the compositions of the present disclosure contain polyvinylpyrrolidone, the polyvinylpyrrolidone ingredient is preferably selected or processed to minimize peroxide content. Freshly produced batches of polyvinylpyrrolidone are preferred over aged batches. Additionally, particularly in cases where the composition will contain greater than 0.5% polyvinylpyrrolidone, the polyvinylpyrrolidone ingredient should be thermally treated (i.e., heated to a temperature above room temperature) prior to mixing with olopatadine in order to reduce the amount of peroxides in the polyvinylpyrrolidone ingredient and minimize the effect of peroxides on the chemical stability of olopatadine. While thermally treating an aqueous solution of polyvinylpyrrolidone for prolonged periods will substantially reduce the amount of peroxides, it can lead to discoloration (yellow to yellowish-brown) of the polyvinylpyrrolidone solution. In order to substantially reduce or eliminate peroxides without discoloring the polyvinylpyrrolidone solution, the pH of the aqueous solution of polyvinylpyrrolidone should be adjusted to pH 11-13 before it is subjected to heat. Much shorter heating times are needed to achieve significant reductions in peroxide levels if the pH of the polyvinylpyrrolidone solution is elevated.

One suitable method of thermally treating the polyvinylpyrrolidone ingredient is as follows. First, dissolve the polyvinylpyrrolidone ingredient in purified water to make a 4-6% solution, then raise the pH of the solution to pH 11-13, (an effective range of pH is 11-11.5), then heat to a temperature in the range of 60-121° C., preferably 65-80° C. and most preferably 70-75° C. The elevated temperature should be maintained for approximately 30-120 minutes (preferably 30 minutes). After the heated solution cools to room temperature, add HCl to adjust the pH to 3.5-8, depending upon the target pH for the olopatadine composition.

Particularly for compositions intended to be administered as eye drops, the compositions preferably contain a tonicity-adjusting agent in an amount sufficient to cause the final composition to have an ophthalmically acceptable osmolality (generally 150-450 mOsm, preferably 250-350 mOsm). The ophthalmic compositions of the present disclosure preferably have a pH of 4-8, preferably a pH of 6.5-7.5, and most preferably a pH of 6.8-7.2.

The eye-drop compositions of the present disclosure are preferably packaged in opaque plastic containers. A preferred container for an ophthalmic product is a low-density polyethylene container that has been sterilized using ethylene oxide instead of gamma-irradiation.

With respect to opthamalic injectables, the pharmaceutical compositions of this disclosure are administered to the area in need of treatment by subconjunctival administration. One preferred method of subconjunctival administration to the eye is by injectable formulations comprising the pharmaceutical compositions disclosed herein. Another preferred method of subconjunctival administration is by implantations comprising slow releasing compositions.

Compositions that are delivered subconjunctivally comprise, according to embodiments, an ophthalmic depot formulation comprising an active agent for subconjunctival administration. According to embodiments, the ophthalmic depot formulation comprises microparticles of essentially pure active agent, e.g., the phenylphthalimide analogs disclosed herein, such as Compound 4. The microparticles comprising can be embedded in a biocompatible pharmaceutically acceptable polymer or a lipid encapsulating agent. The depot formulations may be adapted to release all of substantially all the active material over an extended period of time. The polymer or lipid matrix, if present, may be adapted to degrade sufficiently to be transported from the site of administration after release of all or substantially all the active agent. The depot formulation can be liquid formulation, comprising a pharmaceutical acceptable polymer and a dissolved or dispersed active agent. Upon injection, the polymer forms a depot at the injections site, e.g., by gelifying or precipitating.

Solid articles suitable for implantation in the eye can also be designed in such a fashion to comprise polymers and can be bioerodible or non-bioerodible. Bioerodible polymers that can be used in preparation of ocular implants carrying the compositions of the present disclosure include without restriction aliphatic polyesters such as polymers and copolymers of poly(glycolide), poly(lactide), poly(.epsilon.-caprolactone), poly(hydroxybutyrate) and poly(hydroxyvalerate), polyamino acids, polyorthoesters, polyanhydrides, aliphatic polycarbonates and polyether lactones. Illustrative of suitable non-bioerodible polymers are silicone elastomers.

According to implementations, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to cell-specific antigens) can also be used as pharmaceutically acceptable carriers. These can be prepared according to methods known to those skilled in the art, for example, as described in U.S. Pat. No. 4,522,811, which is incorporated by reference herein.

It is advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier.

Toxicity and therapeutic efficacy of such compounds may be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds which exhibit high therapeutic indices are effective. While compounds that exhibit toxic side effects can be used, care should be taken to design a delivery system that targets such compounds to the site of affected location to minimize potential damage to uninfected cells and, thereby, reduce side effects.

The data obtained from the cell culture assays and animal studies can be used in formulating a range of dosage for use in humans. The dosage of such compounds lies preferably within a range of circulating concentrations that include the ED50 with little or no toxicity. The dosage can vary within this range depending upon the dosage form employed and the route of administration utilized. For any compound used in the method of the disclosure, the therapeutically effective dose can be estimated initially from cell culture assays. A dose can be formulated in animal models to achieve a circulating plasma concentration range that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

As defined herein, a therapeutically effective amount of the active compound (i.e., an effective dosage) may range from about 0.001 to 100 g/kg body weight, or other ranges that would be apparent and understood by artisans without undue experimentation. The skilled artisan will appreciate that certain factors can influence the dosage and timing required to effectively treat a subject, including but not limited to the severity of the disease or disorder, previous treatments, the general health or age of the subject, and other diseases present.

### EXAMPLES

A more complete understanding of the present disclosure can be obtained by reference to the following specific examples and figures. The examples and figures are described solely for purposes of illustration and are not intended to limit the scope of the disclosure. Changes in form and substitution of equivalents are contemplated as circumstances may suggest or render expedient. Although specific terms have been employed herein, such terms are intended in a descriptive sense and not for purposes of limitations. Modifications and variations of the disclosure as hereinbefore set forth can be made without departing from the spirit and scope thereof, and, therefore, only such limitations should be imposed as are indicated by the appended claims.

### Example 1

### Materials and Methods

Cell culture: All cell culture media and supplements were purchased from Cellgro unless otherwise indicated. Human Umbilical Vein Endothelial Cells (HUVEC) were obtained from American Type Culture Collection and grown in the EBM-MV2 medium (Clonetics). Bovine Retinal Endothelial Cells (BREC) and pericytes were isolated according to a modified method as described previously (Wong, et al. Investig. Opthalmol. Vis. Sci. 1987, 28: 1767-1775). Twelve bovine eyes were obtained from a local slaughterhouse (Country Home Meats). The retinas were removed and washed four times in DMEM. Subsequently retinas were homogenized and centrifuged at 400×g for 10 min. The resultant pellet was resuspended in an isolation medium (DMEM with 100 IU/ml penicillin, 100 μg/ml streptomycin and 250 ng/ml amphotericin). Microvessels were trapped on an 85 μm nylon mesh (Locker Wire Weavers LTD) and transferred to a petri dish (Falcon) containing 10 ml of an enzyme cocktail which consisted of 600 μg/ml DNase I (Sigma), 165 μg/ml collagenase (Sigma) and 700 μg/ml Pronase E (EMD) and were incubated at 37° C. for 20 min. The resultant vessel fragments were trapped on a 53 μm nylon mesh (Locker Wire Weavers LTD), washed with the isolation medium and centrifuged at 400×g for 5 min. For selective culture of pericytes, the resultant pellet was resuspended in 10 ml of the pericyte growth medium and transferred into 75-cm² plastic tissue culture flasks (BD Biosciences). For selective culture of BRCECs, the resultant pellet was resuspended in 10 ml of the BRCEC growth medium and transferred into 75-cm² collagen-coated plastic tissue culture flasks (BD Biosciences). The BRCEC growth medium consisted of DMEM supplemented with 10% human serum, 1% glutamine, 1 mg/ml insulin, 550 μg/ml transferring, 670 ng/ml selenium, 100 IU/ml penicillin, 100 μg/ml streptomycin, 250 ng/ml amphotericin, 90 μg/ml heparin (Sigma) and 15 μg/ml endothelial cell growth supplement (Upstate). Cells were cultured at 37° C. and 5% CO₂ with regular medium change every 3 days. Confluence cultures were passaged by detaching the cells with 0.25% trypsin and plated at a split 1:3. Purity of BRCECs and pericytes were confirmed by binding of Dil-Ac-LDL (Biomedical Technologies Inc) to LDL receptor on the surface of BRCECs and immunolabeling with anti-smooth muscle antibody (Sigma), respectively. At passage 2, BRCECs and pericytes were stored in a liquid nitrogen tank for future use.

MTT assay: Cells were seeded at a density of 5×10⁴ cells per well in 400 μl of growth medium in triplicate in 24-well plates (Nalge Nunc) or gelatin-coated 24-well plates. Twenty-four hours after seeding, the growth medium was replaced by a medium containing 1% FBS, with or without different concentrations of thalidomide or phenylphthalimide analogs. After the cells were treated for 48-72 h, MTT was added to a final concentration of 0.5 mg of medium per ml and incubated for 4 h at 37° C. in 5% CO₂. An equal volume of solubilizer buffer is then added, following the protocol recommended by the manufacturer (Roche Molecular Biochemicals), the cells will be incubated overnight at 37° C. in 5% CO₂. The absorbance of the formazen product was measured at a wavelength of 570 nm, with 750 nm as the (subtracted) reference wavelength.

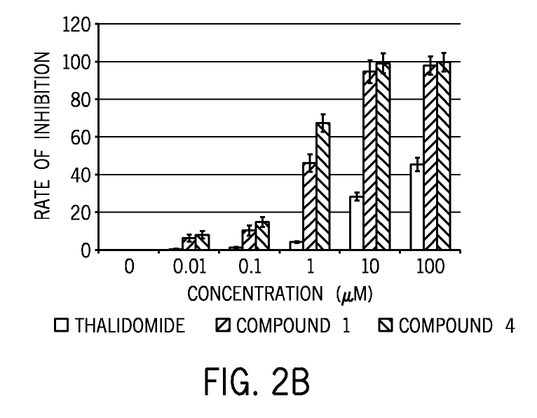

Endothelial cell migration assay: The fluorescence-based endothelial cell invasion assay used a BD Matrigel™ and BD Falcon™ HTS FluoroBlok™ (BD Biosciences) 24-Multiwell Insert System (FIG. 2A). The insert system consisted of fluorescence-blocking 3 μm PET membrane, which blocks light transmission at wavelengths 490-700 nm, sealed to multiwell inserts. This made it possible to directly measure fluorescent signal from cells that had undergone invasion through Matrigel to the bottom side of inserts by using signal from cells that had undergone invasion through Matrigel to the bottom side of inserts by using bottom reading mode of a fluorometer. In this assay system, HUVECs were allowed to invade in the absence (control) or presence of VEGF (4 ng/ml) with varying concentrations (0.01-100 μM) of Compounds 1, Compound 4 and thalidomide in the bottom. Cells were allowed to invade for 22±1 hours. Cells were labeled post invasion with Calcein AM (4 μg/ml) and measured by detecting the fluorescence of cells that invaded through the BD Matrigel™ Matrix with an Applied Biosystems CytoFluor® 4000 plate reader at 485 nm excitation and 530 nm emission.

Chicken chorioallantoic membrane (CAM) assay: The fertile Leghorn chicken eggs were incubated in a humidified environment at 37.5° C. for 10 days. The human VEGF-165 and basic fibroblast growth factor (bFGF) (200 ng each) were then added to saturation to a microbial testing disk and placed onto the CAM by breaking a small hole in the superior surface of the egg. Anti-angiogenic compounds were then added 8 hr after the VEGF/bFGF at saturation to the same microbial testing disk, and the embryos were incubated for an additional 40 h. CAMs were then removed, quickly fixed with 4% paraformaldehyde in PBS, placed onto Petri dishes, and digitized images taken at 7.5× using a Nikon dissecting microscope and Scion Imaging system. A 1×1-cm grid was then added to the digital CAM images and the average number of vessels within 5-7 grids counted as a measure of vascularity.

Induction of oxygen-induced retinopathy (OIR): Induction of OIR followed the procedure as described by Smith et al (Smith, et al. Invest. Opthalmol. Vis. Sci. 1994, 35: 101-111) with some modifications. Briefly, Newborn Brown Norway (BN) rats (Charles River Laboratories) at postnatal day 7 (P7) were exposed to hyperoxia (75% O₂) for 5 days (P7-12) and then returned to normoxia (room air) to induce retinopathy.

Induction of diabetes by streptozotocin (STZ): BN rats (8 weeks of age) were given a single intraperitoneal injection of fresh made streptozotocin (STZ) (Sigma, 50 mg/kg in 10 mM of citrate buffer, pH 4.5) following an overnight fasting. Control rats received an injection of citrate buffer alone. Blood glucose levels were checked at 24 hours following the last STZ injection and once a week thereafter, and only the animals with glucose levels higher than 350 mg/dl were considered diabetic. Rats with hyperglycemia for 2 weeks were used for these experiments.

Intravitreal injection of compounds: Thalidomide and the phenylphthalimide analogs Compounds 1, 2 and Compound 4 were dissolved in vehicle (BN rat serum) and sterilized by filtration. OIR and STZ-diabetic BN rats received an intravitreal injection of 0.5-2.0 μg/eye of (5 μl/eye, 0.1-0.4 mg/ml in BN rat serum) of thalidomide, Compounds 1, 2 or Compound 4 into the right eye and the equal volume of the BN rat serum into the left eye.

Retinal angiography with high-molecular-weight fluorescein: High molecular weight fluorescein-dextran was used in retinal angiography as described by Smith et al (Smith, et al. Invest. Opthalmol. Vis. Sci. 1994, 35:101-111). Briefly, animals were anesthetized with ketamine (100 mg/kg of body weight) plus acepromazine (5 mg/kg of body weight) and then perfused through the left ventricle with 50 mg/ml of high molecular weight fluorescein-dextran in PBS. The eyes were marked for orientation, enucleated, and fixed in 4% paraformaldeyde for 3-24 h. Several incisions were made and the retinas were flat-mounted on a gelatin-coated slide. The vasculature was then examined under a fluorescent microscope. Both the total retinal area and the area of the avascular regions were measured using a computerized image-analysis system and averaged within each group.

Measurement of vascular permeability: Vascular permeability was quantified by measuring leakage of FITC-albumin or Evans blue dye-albumin complex from the blood vessels into the retina as described (Xu, et al. Invest. Opthalmol. Vis. Sci. 2001, 42:789-794), with some modifications. Briefly, FITC-albumin was injected through the femoral vein and circulated for 2 h. The rats were then perfused via the left ventricle. The retinas were carefully dissected and homogenized. The concentrations of FITC-albumin were measured in a fluorometer and normalized by the total protein concentration in each retina and by plasma concentration of FITC-albumin.

Evans blue dye (Sigma) was dissolved in 0.9% saline (30 mg/ml), sonicated for 5 min and filtered through a 0.45-μm filter (Millipore). The rats were then anesthetized, and Evans blue (30 mg/kg) was injected over 10 s through the femoral vein using a glass capillary under microscopic inspection. Evans blue non-covalently binds to plasma albumin in the blood stream. Immediately after Evans blue infusion, the rats turned visibly blue, confirming their uptake and distribution of the dye. The rats were kept on a warm pad for 2 h to ensure the complete circulation of the dye. Then the chest cavity was opened, and the rats were perfused via the left ventricle with 1% paraformaldehyde in citrate buffer (pH 4.2) which was pre-warmed to 37° C. to prevent vasoconstriction. The perfusion lasted 10 min under the physiological pressure of 120 mmHg, in order to clear the dye from the vessel. Immediately after perfusion, the eyes were enucleated and the retinas were carefully dissected under an operating microscope. Evans blue dye was extracted by incubating each sample in 150 μl of formamide for 18 h at 70° C. The extract was centrifuged (Beckman) at 70,000 rpm (Rotor type: TLA 100.3) for 20 min at 4° C. Absorbance was measured using 100 μl of the supernatant at 620 nm by using Spectrophotometer DU800 (Beckman). The concentration of Evans blue in the extract was calculated from a standard curve of Evans blue in formamide and normalized by the total protein concentration in each sample. Results were expressed in mg of Evans blue per mg of total protein content.

Immunolabeling: Cultured cells were immediately fixed in 4% paraformaldehyde in 1×PBS for 10 min, washed in PBS three times for 5 min, and blocked in 0.5% BSA for 20 min. Washing cells in PBS three times before and after a 1 hr primary antibody incubation was followed by staining for 1 hr with secondary antibodies. The immunolabeling signals were subsequently detected by incubating cells with FITC or Texas red-conjugated secondary antibodies (Jackson Immunoresearch). Coverslips were washed in PBS and stained with 0.2 μg/ml DAPI prior to mounting. Fluorescent images were collected on a Zeiss fluorescent microscope or a Zeiss 510 confocal laser scanning microscope equipped with an argon-krypton laser.

Western blotting: Proteins were extracted by incubating in a lysis buffer. Equal amounts of proteins from different samples were separated by SDS-PAGE for Western blot analyses using an antibody directed against VEGF. Immunobloting signals were visualized by conversion of SuperSignal West Pico Chemiluminescent Substrate (Pierce).

Electroretinogram (ERG) recording: Full-field ERGs were recorded by Espion E² ERG system (Diagnosys LLC) as described previously (Rohrer, Journal of Neuroscience, 1999, 19: 8919-8913) by two protocols: (A) 10 ms flashes of increasing light intensities under scotopic and photopic conditions, and (B) 2 Hz flicker ERG under photopic conditions. BN rats received an intravitreal injection of Compound 4 (2.0 μg/eye, 5 μl/eye of 0.4 mg/ml in BN rat serum) or equal amount of BN rat serum, respectively. At various intervals after injection, the peak a-wave amplitude was measured from baseline to the initial negative-going voltage, whereas peak b-wave amplitude was measured from the trough of the a-wave to the peak of the positive b-wave. Flicker amplitudes were measured from the preceding trough to the peak of the flicker response. Data was expressed as mean±SD and compared between the compound-injected eyes and control eyes by the paired Student's t-test.

Histological analysis of the retina: To test the potential ocular toxicity of Compound 4, 8 week old normal BN rats received an intravitreal injection of Compound 4 (2.0 μg/eye, 5 μl/eye of 0.4 mg/ml in BN rat serum) or equal amount of BN rat serum, respectively. At various intervals after injection, the animals were sacrificed. The eyes then were removed, fixed in 4% formaldehyde, embedded in paraffin, and cut into 6-μm sections containing the whole retina. Paraffin-embedded sections were stained with hematoxylin-eosin (HE) and were examined.

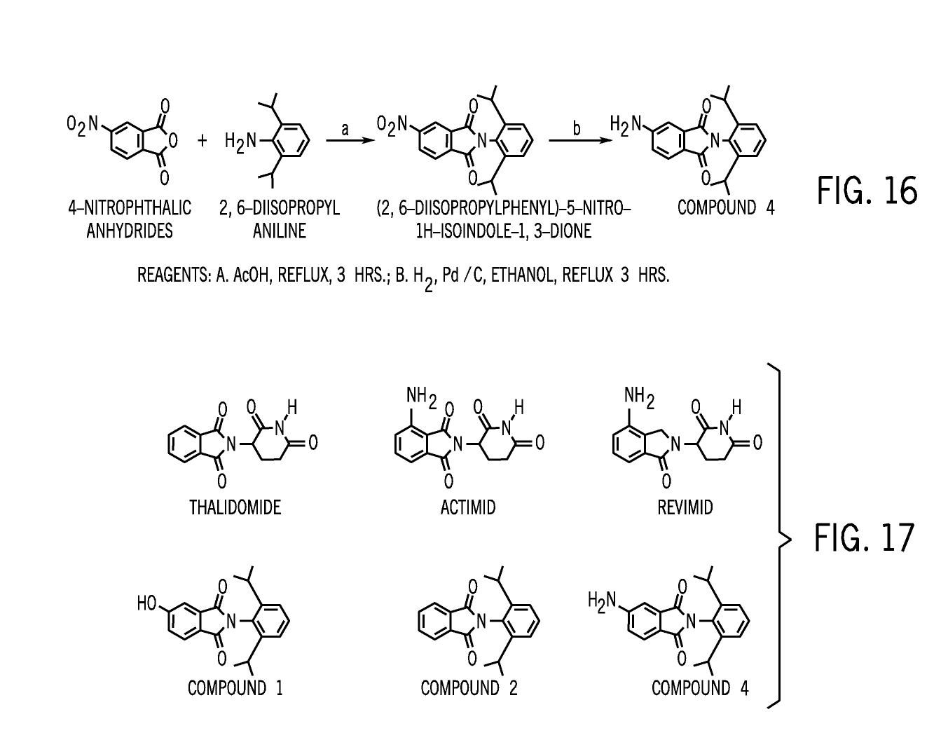

A series of novel phenylphthalimide analogs have been designed, synthesized and experimentally tested. The discoveries and results of the experiments are included below. FIG. 16 is a diagram showing the route of synthesis for Compound 4. ¹H-NMR (250 MHz, CDCl₃) δ1.08 (12H, d, J=6.80 Hz), 2.50 (2H, hept, J=6.80 Hz), 6.75 (1H, dd, J=1.98 Hz, 8.25 Hz), 6.87 (1H, d, J=1.98 Hz), 7.16 (2H, d, J=7.85 Hz), 7.31 (1H, t, J=7.85 Hz), 7.46 (1H, d, J=8.25 Hz). mp 252-253° C. (lit. 253-254° C.). FIG. 17 is a diagram of the various chemical structures of thalidomide, actimid, revimid, Compounds 1, 2, and 4.

### Example 2

### Compound 4 was Found to be Substantially More Potent than Thalidomide and the Other Two Analogs in Inhibition of Proliferation of Endothelial Cells

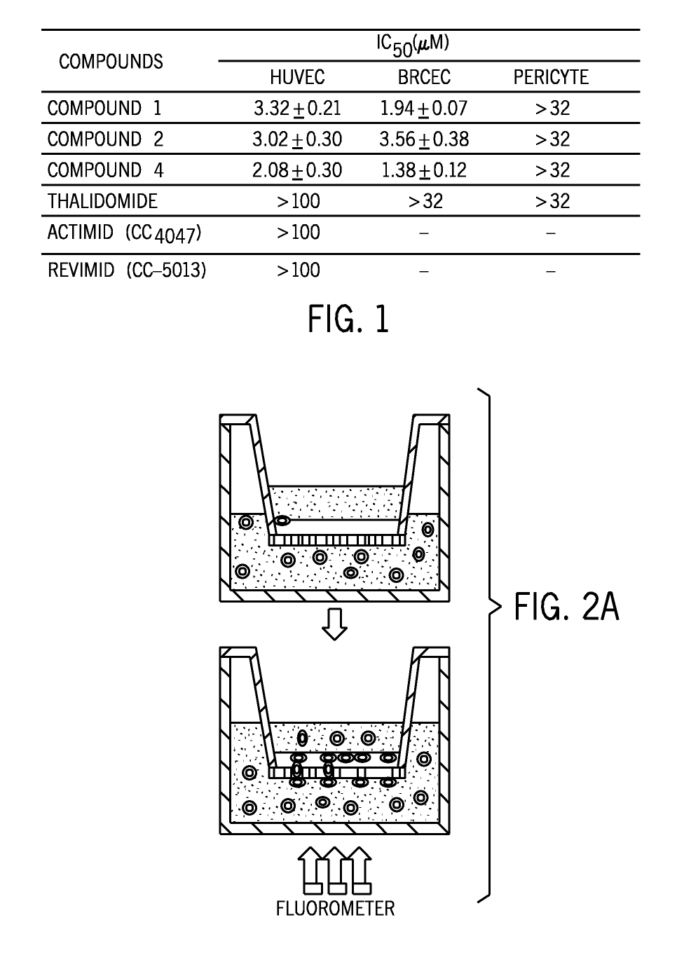

Primary endothelial cells (HUVEC and BRCEC) and pericytes were treated with various concentrations of the compounds for 3 days. Viable cells were quantified using MTT assay and IC₅₀ of each compound was calculated (mean±SD, n=3, FIG. 1). The IC₅₀ values represent the means and SE of 3 independent experiments. Compounds 1, 2 and Compound 4 inhibited the proliferation of endothelial cells in a dose-dependent manner with an IC₅₀ of 3.3, 3.0 and 2.0 μM, respectively, for HUVECs and 1.94, 3.56 and 1.83 μM, respectively, for BRCECs. thalidomide had weaker effects with IC₅₀>100 μM in HUVECs and with IC₅₀>32 μM in BRCECs (FIG. 1). Existing phenylphthalimide analogs, Actimid (CC4047) and Revimid (CC-5013), had weaker effects with IC₅₀>100 μM in HUVECs. Under the same conditions, these compounds did not significantly inhibit pericyte growth, suggesting specific inhibition to endothelial cells. These results indicated that Compound 4 had more potent anti-angiogenic effects than the other 2 compounds and thalidomide.

FIG. 1 is a table comparing the effect of thalidomide and the new phenylphthalimide analogs on cell proliferation.

### Example 3

### Compound 4 was Found to have a More Potent Inhibitory Effect than Thalidomide on Migration of HUVEC

The effect of Compound 1, Compound 4, and thalidomide on endothelial migration was evaluated using in vitro migration (invasion) assay. The advantage of this assay is that it can be amended to high-throughput screening. It is a fluorescence-based endothelial cell invasion assay system. This assay system is based on BD Matrigel™ and BD Falcon™ HTS FluoroBlok™ (BD Biosciences, Bedford, Mass.) 24-Multiwell Insert System (FIG. 2A). The insert system consists of fluorescence-blocking 3 μm PET membrane, which blocks light transmission at wavelengths 490-700 nm, sealed to multiwell inserts (FIG. 2A). This makes it possible to directly measure fluorescent signal from cells that have undergone invasion through Matrigel to the bottom side of inserts by using signal from cells that have undergone invasion through Matrigel to the bottom side of inserts by using bottom reading mode of a fluorometer. In this assay system, human endothelial cells are allowed to invade and are then labeled with fluorescent dye Calcein AM before quantification on a fluorometer. Compound 1 and Compound 4 inhibited the endothelial cell migration and showed the dose response curves with an IC₅₀ of 1 μM and <1 μM. Thalidomide, on the other hand, exhibited an IC₅₀ of >100 μM, as shown in FIG. 2B.

FIG. 2 compares the inhibition of endothelial cell (HUVEC) migration by Compound 1, Compound 4, and thalidomide. FIG. 2A shows a schematic illustration of the Endothelial Cell Invasion assay system. In FIG. 2B, cells were seeded at 5×10⁴/insert in EBM-2 containing 0.1% BSA in multi-well inserts. The assembled assays were allowed to proceed for 6 hours. The results are expressed as percent inhibition of migration as compared to control (no inhibitor). Data represents the average of 3 experiments, each run in triplicate. The bars represent mean±SD.

### Example 4

### Compound 4 was Found to Inhibit Tube Formation from Endothelial Cells

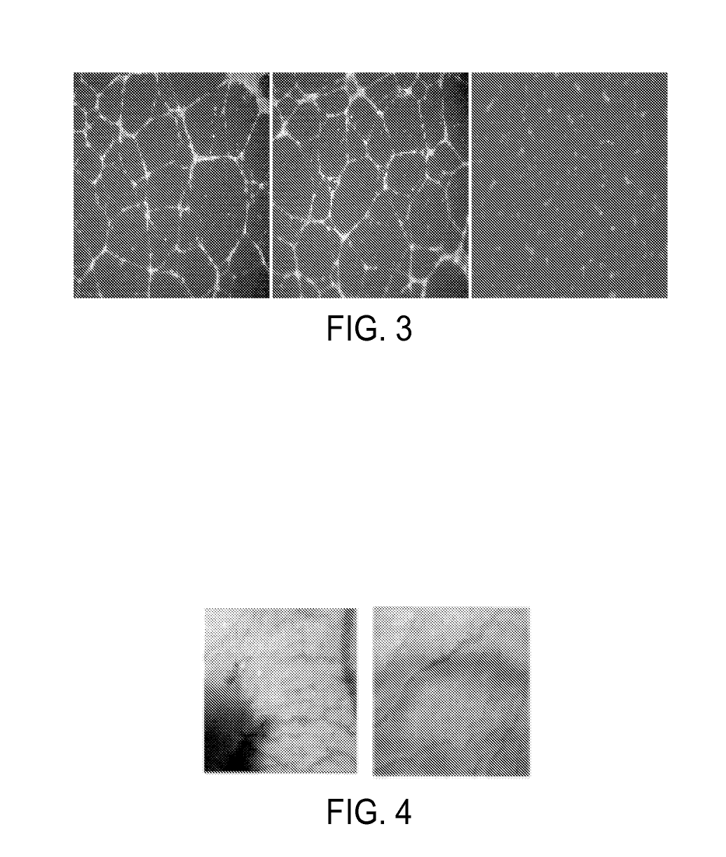

Eight-well slide chambers were coated with matrigel and at 37° C. and 5% CO₂ for 30 min. HUVECs were then seeded at 30,000 cells/well in EGM-II containing either vehicle (0.5% DMSO), 5 μM of Compound 4 or thalidomide and incubated at 37° C. and 5% CO₂ for 16 h. After incubation, slides were washed in PBS, fixed in 100% methanol for 10 s, and stained with DiffQuick solution II for 2 min. To analyze tube formation, each well was digitally photographed using a ×2.5 objective. The tube formation assay showed the qualitative representative images of the potency of Compound 4 on inhibition of tube formation. On the contrary, thalidomide did not show any inhibitory activity as shown in FIG. 3.

FIG. 3 shows the effect of Compound 4 on tube formation. Representative images were captured after incubation of vehicle, thalidomide and Compound 4 for 16 h. Compound 4 was shown to effectively inhibited tube formation.

### Example 5

### Compound 4 was Found to be More Potent than Thalidomide and Compounds 1 and 2 in Inhibiting Vascular Formation in the Cam Assay

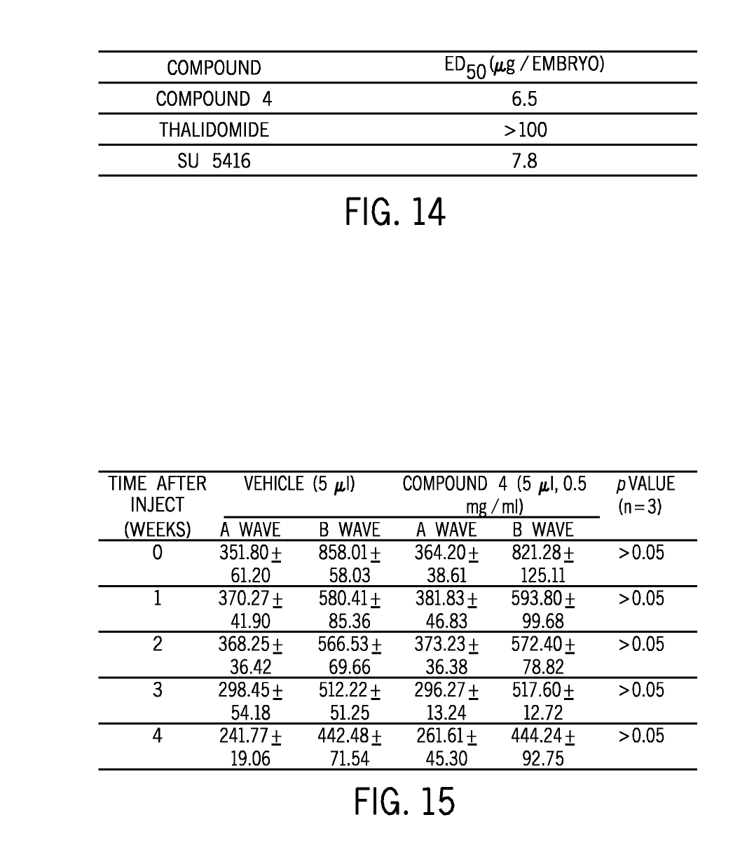

CAM assay was used for in vivo anti-angiogenic studies. The fertile leghorn chicken eggs were allowed to incubate in a humidified environment at 37.5° C. for 10 days. The human VEGF-165 and bFGF (200 ng each) were then added to saturation to a microbial testing disk and placed onto the CAM by breaking a small hole in the superior surface of the egg. Anti-angiogenic compounds were then added 8 hours after the VEGF/bFGF at saturation to the same microbial testing disk and embryos allowed to incubate for an additional 40 hours. After 48 hr, CAMs were removed, quickly fixed with 4% paraformaldehyde in PBS, placed onto Petri dishes, and digitized images taken at 7.5× using a Nikon dissecting microscope and Scion Imaging system. A 1×1-cm grid was then added to the digital CAM images and the average number of vessels within 5-7 grids counted as a measure of vascularity. FIG. 4 shows a representative CAM treated with VEGF-165/bFGF for 48 hr and a CAM treated with VEGF/bFGF and 5 μg of Compound 4 for 48 hr. VEGF/bFGF induced CAM blob vessel formation. At 5 μg/embryo, Compound 4 was able to inhibit CAM blood vessel formation induced by VEGF/bFGF. Compound 4 has an ED₅₀ of 6.5 μg/embryo, while thalidomide has an apparent ED₅₀ of >100 μg/embryo, suggesting Compound 4 inhibited blood vessel formation in the CAM assay (FIG. 14).

FIG. 4 shows the effect of Compound 4 on blood vessel formation in CAM assay. The left panel in FIG. 4 represents a CAM treated with 200 ng VEGF-165/bFGF for 48 hr. The right panel in FIG. 4 is a representation of a CAM assay treated with 200 ng of VEGF-165/bFGF and 5 μg/embryo of Compound 4.

FIG. 14 is a table comparing the effect of Compound 4 on blood vessel formation. Thalidomide and SU5416 on dose in μg/embryo of compound necessary to reduce the blood vessel number to 50% that of the VEGF/bFGF alone group, a level of blood vessels similar to the untreated “control” group. Thus thalidomide alone has an apparent ED₅₀ of >100 μg/embryo, Compound 4 and SU5416 has an apparent ED50 of 6.5 and 7.8 μg/embryo, respectively. Data represent mean±SD of 8-16 samples from 2-3 separate experiments.

### Example 6

### Phenylphthalimide Analogs were Found to Suppress Hypoxia-Induced HIF-1α Production in PC-3 Prostate Cancer Cells

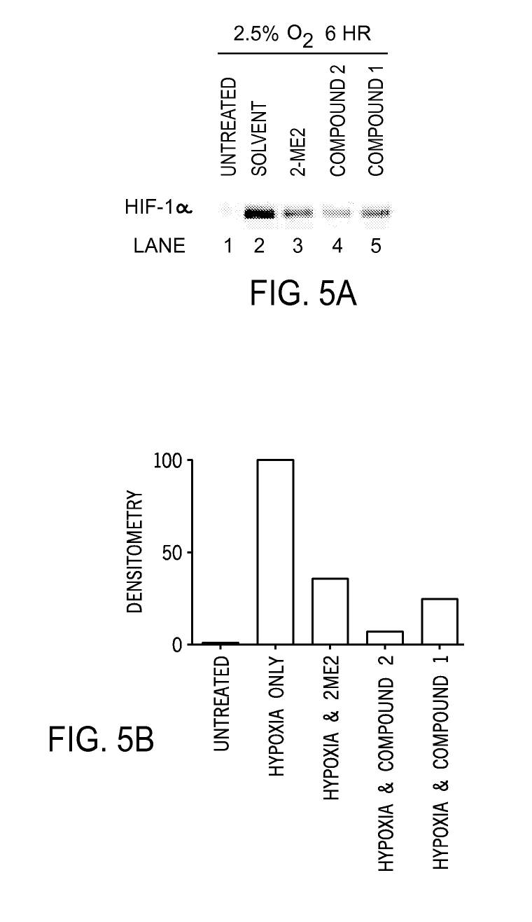

Suppression of hypoxia-induced HIF-1α expression by Compound 1, Compound 2 and 2ME2 (positive control) was tested in PC-3 prostate cancer cells. Cells were exposed to 10 μM (containing 0.1% DMSO) of inhibitors or DMSO alone as control overnight. HIF-1α expression in the PC-3 prostate cancer cell treated by hypoxia and compounds was analyzed by western blotting (FIG. 5A). Both compound 1 and 2 significantly suppress hypoxia-induced HIF-1α expression by 79-90% (FIG. 5B).

FIG. 5 shows the effect of phenylphthalimide analogs on HIF-1α expression. HIF-1α in the PC-3 prostate cancer cell treated by hypoxia and compounds was analyzed by western blot (FIG. 5A). Quantitative analysis showed both compound 1 and 2 suppressed hypoxia-induced HIF-1α expression (FIG. 5B).

### Example 7

### Compound 4 was Found to Down-Regulate the Expression of VEGF in the Retina of OIR Rats

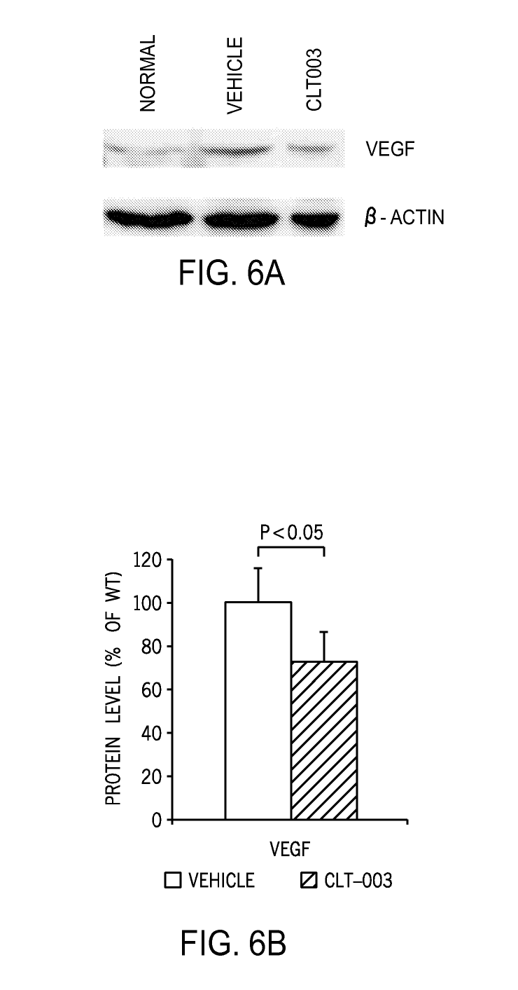

VEGF is believed to play a critical role in diabetic macular edema. HIF-1α regulates transcriptional activation of VEGF in response to hypoxia. The tested phenylphthalimide analogs significantly suppressed hypoxia-induced HIF-1α expression in vitro studies, suggesting that these compounds may reduce retinal vascular leakage through VEGF signaling. To address the hypothesis, the expression of VEGF in the Compound 4-injected OIR rats was determined. Proteins of retinas from normal rats, vehicle-treated and Compound 4-treated OIR rats were extracted by incubating and sonicating in lysis buffer. Equal amounts of proteins from each samples were separated by SDS-PAGE for Western blot analyses using antibody directed against VEGF. Immunoblotting signals were visualized by conversion of SuperSignal West Pico Chemiluminescent Substrate (Pierce). The result has shown that the expression of VEGF decreased in retina of Compound 4-treated OIR rat (FIG. 6).

FIG. 6 shows that Compound 4 (Compound 4) down-regulated the expression of VEGF in the retina of OIR rats. VEGF levels in the retinas from normal rats, vehicle-treated and Compound 4-treated OIR rats was determined by Western blotting (FIG. 6A). FIG. 6B shows the quantitative analysis of VEGF expression. The lane labeled “Normal” represents normal BN rat, “Control” represents intravitreal injection of 5 μl BN rat serum into the left eye, “Compound 4” represents intravitreal injection of 5 μl Compound 4 (0.8 mM in BN rat serum) into the right eye.

### Example 8

### Compound 4 was Found to have a More Potent Effect on Retinal Vascular Leakage in OIR Rats after an Intravitreal Injection

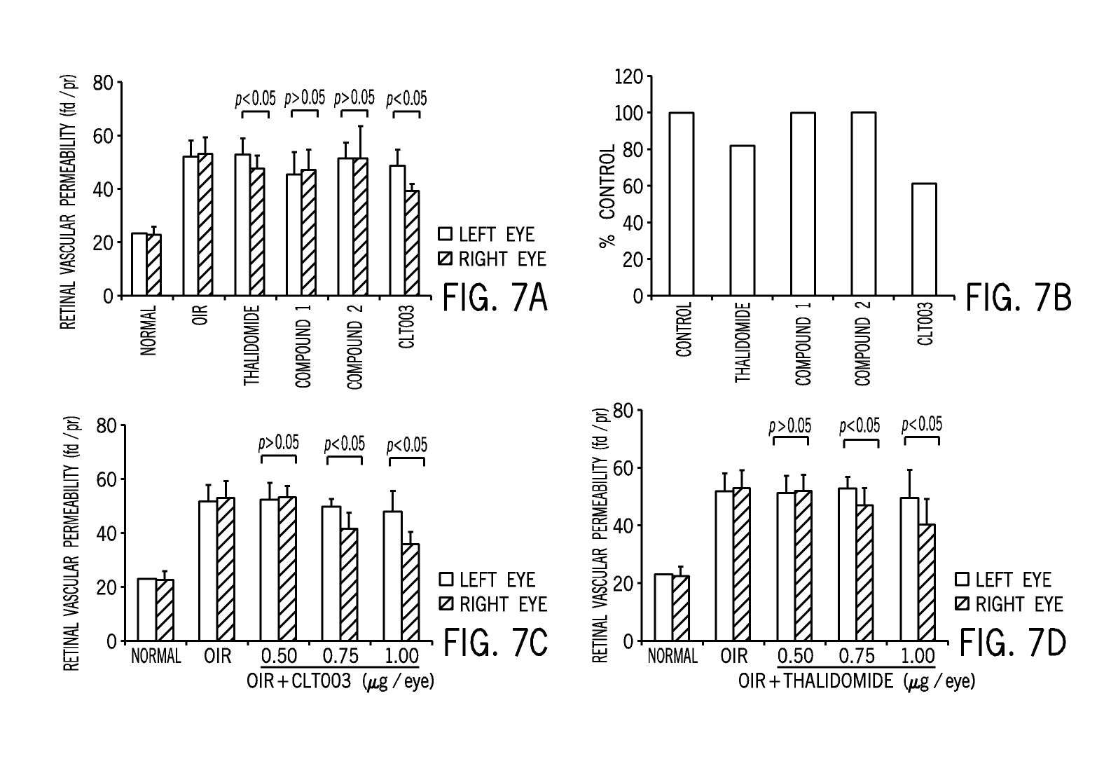

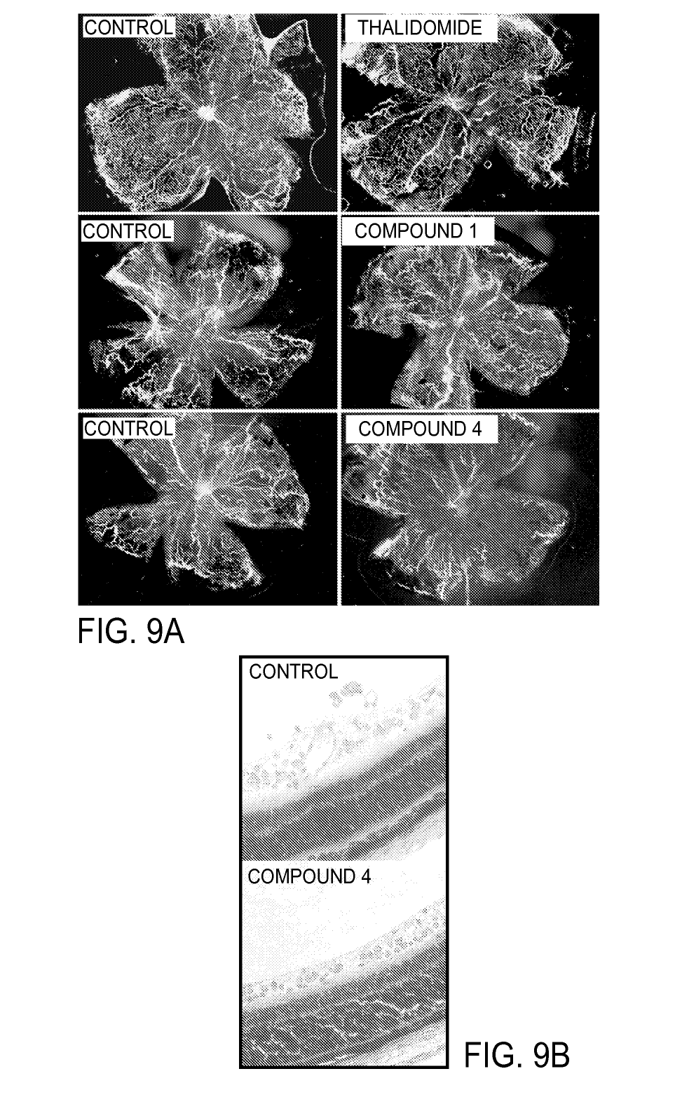

To induce OIR, BN rats at postnatal day 7 (P7) were exposed to hyperoxia (75% O₂) for 5 days (P7-P12) and then returned to normoxia. Normal control rats were kept in room air. At P14, the OIR BN rats received an intravitreal injection of 5 μl (0.8 mM in BN rat serum)/eye of thalidomide, Compound 1, Compound 2, or Compound 4 into the right eye and same volume of the BN rat serum into the left eye. Retinal vascular leakage was measured using FITC-labeled albumin as tracer. Normal non-OIR BN rats (n=6) served as baseline at P16. At P16, retinal vascular leakage decreased in the thalidomide-treated eyes to 82% of the contralateral eyes injected with vehicle (paired t test, P<0.05, n=6). Compound 4 decreased the retinal vascular leakage to 61% of the contralateral control (paired t test, P<0.05, n=6). At the same concentration, Compounds 1 and 2 did not significantly reduce the retinal vascular leakage (FIGS. 7A and 7B). Fluorescein angiography showed that Compound 4 had weak effect on retinal NV at the dose used (FIG. 9), suggesting that Compound 4 induced reduction of retinal vascular leakage is more potent than its effect on retinal NV.

To determine if the effect of Compound 4 on retinal vascular leakage was dose-dependent, the OIR rats at P14 received a single injection of Compound 4 with doses of 0.5, 0.75 and 1.0 μg/eye (5 μl of 0.10, 0.15 and 0.20 mg/ml). Compound 4 and thalidomide significantly reduced vascular leakage at doses of 0.75 and 1.0 μg/eye (p<0.05, n=6) but not at the dose of 0.5 μg/eye (FIGS. 7C and 7D), indicating a dose-dependent effect on vascular leakage in OIR rats.

FIG. 7 compares the effect of Compounds 1, 2, 4 (Compound 4) and thalidomide on retinal vascular leakage in OIR rats. In FIG. 7A, OIR rats received an intravitreal injection of 5 μl (0.8 mM in BN rat serum)/eye of thalidomide, Compounds 1, 2, or 4 in the right eye and the same volume of the vehicle in the left eye at P14. Vascular leakage was measured using the FITC-labeled albumin leakage method at P16 and expressed as fd/pr of protein in the retina (mean±SD, n=6). Each of the experimental group was compared with contralateral eye by Student's t test. Retinal vascular leakage in normal non-OIR rats at age of P16 were used as baseline at P16. In FIG. 7B, vascular leakage in the compound-injected eyes was expressed as a percentage of average vascular leakage in the vehicle-injected contralateral eyes. For the control, the average vascular leakage in vehicle-treated retinas was used as 100%. The thalidomide and Compound 4 reduced retinal vascular leakage by 18% and 40%, respectively (n=6). In FIGS. 7C and 7D, OIR rats received an intravitreal injection of Compound 4 or thalidomide with doses as indicated at P14. Permeability was measured at P16 and expressed as fd/pr of protein in the retina (mean±SD, n=6). Each of the experimental group was compared with the vehicle control by the paired Student's t test. “Normal” is represented as the permeability in normal rats at P16.

### Example 9

### Compound 4 was Found to have a More Potent Effect on Retinal Vascular Leakage in STZ-Diabetic Rats

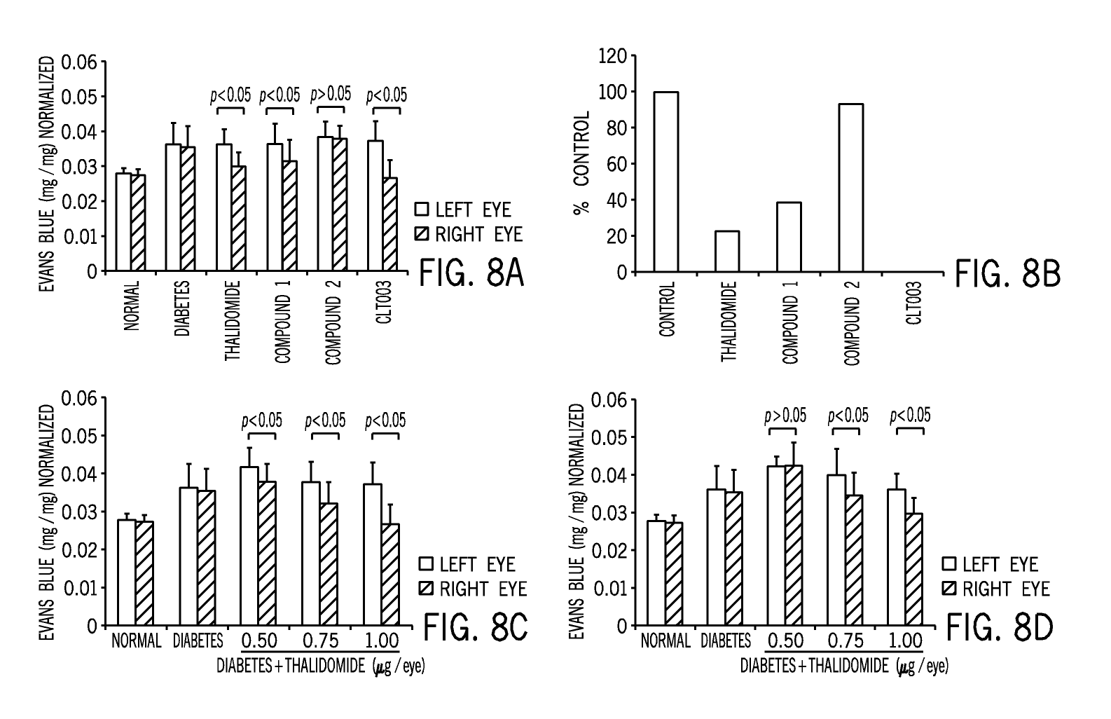

Diabetes was induced by injection of STZ (50 mg/kg, i.v.) into adult BN rats after overnight fasting. Blood glucose levels were monitored at the second day after the injection and once a week thereafter. Rats with glucose levels above 350 mg/dl were considered as diabetic and used for the study. thalidomide, Compounds 1, 2 and 4 were separately injected into the vitreous space (5 μl, 0.8 mM in BN rat serum) of the right eye of STZ-diabetic rats 2 wks after the induction of diabetes. At 48 h after the injection, retinal vascular leakage was measured using the Evans blue-albumin leakage method. The result showed that the eyes injected with thalidomide, Compound 1, and Compound 4 had a significant reduction in vascular leakage in the retinas, compared to the contralateral eyes injected with the vehicle (P<0.01, n=6) (FIG. 8A). thalidomide reduced vascular leakage by 77%, Compound 1 reduced vascular leakage by 61%, and Compound 4 reduced vascular leakage by almost 100% (FIG. 8B), to normal level (baseline), suggesting that Compound 4 completely blocks the retinal vascular leakage. To determine the time course of the effect of Compound 4 after intravitreal injection, OIR rats received 5 μl (0.8 mM in BN rat serum)/eye of Compound 4 into the right eye at P14. 24 h and 48 h after administration, retinal vascular permeability measurements showed that the Compound-injected eye had completely been blocked in comparison with the control of the contralateral eye.

To determine the dose-response relationship of the effect of Compound 4 and thalidomide, the STZ-diabetic rats received an intravitreal injection of Compound 4 and thalidomide with doses of 0.5, 0.75 and 1.0 μg/eye (5 μl/eye of 0.10, 0.15 and 0.20 mg/ml), respectively. Two days after the injection, Compound 4, at all of these doses significantly reduced vascular permeability in the retina, when compared to the vehicle control (P<0.05, n=6) (FIG. 8C). However, thalidomide showed an inhibitory effect only at the doses of 0.75 and 1.0 μg/eye (P<0.05, n=6), but not at 0.5 μg/eye (p>0.05, n=6) (FIG. 8D). This observation indicates that Compound 4 has more potent effect on reducing retinal vascular leakage not only in the OIR model but also in the experimental diabetes model, compared to thalidomide and the other compounds.

FIG. 8 compares the effect of thalidomide, Compounds 1, 2 and 4 (Compound 4) on retinal vascular leakage in STZ-diabetic rats. In FIG. 8A, two weeks after the induction of diabetes by STZ, diabetic rats received an intravitreal injection of 5 μl (0.8 mM in BN rat serum) per eye of thalidomide, Compounds 1, 2 or Compound 4 into the right eye and the same volume of the vehicle into the left eye. Retinal vascular permeability in the retina was measured by Evans blue-albumin leakage method, 2 days after the injection and normalized by the total protein concentration in the retina and the Evans blue concentration in the blood (mean±SD, n=6). Each of the experimental group was compared with contralateral eyes by Student's t test. Vascular permeability in non-diabetic rats was used as baseline of permeability. “Normal” is represented as the permeability in normal rats at P16. In FIG. 8B, vascular leakage in the compound-injected eyes was expressed as a percentage of that in the vehicle-injected eyes. As the control, STZ-diabetic rats were injected with the vehicle. thalidomide, Compounds 1, 2 and Compound 4 reduced retinal vascular leakage by 77%, 61% and 100%, respectively (n=6). In FIGS. 8C and 8D, STZ-diabetic rats received an intravitreal injection of Compound 4 or thalidomide with doses as indicated 2 weeks after the induction of diabetes. Permeability was measured 48 h after injection and expressed as mg of Evans blue per mg of protein in the retina (mean±SD, n=6). Each of the experimental group was compared with the vehicle control by the paired Student's t test. “Normal” is represented as the permeability in normal rats.

### Example 10

### Compound 4 was Found to have an Inhibitory Effect on Retinal NV in the OIR Model

Newborn BN rats were exposed to 75% oxygen from age P7 to P12. The rats were then kept in room air for 4 days to allow partial formation of retinal NV. At age P16 when retinal NV has formed partially, OIR rats received a single intravitreal injection of thalidomide and Compounds 1, 2, and Compound 4 of 1.0 μg/eye (5 μl/eye of 0.2 mg/ml in BN rat serum) into the vitreous of the right eye and the vehicle (5 μl BN rat serum) into the left eye for control. Retinal NV was evaluated at age P20 by fluorescein angiography in flat-mounted retinas. The retinal vasculature was visualized under a fluorescent microscope and compared with that in the contralateral control eye (FIG. 9A). The neovascular events were observed on eye sections (FIG. 9B). Results displayed that Compound 4 partly inhibited the retinal NV in OIR rats, while Compound 1, Compound 2, and thalidomide lacked significant inhibition of retinal NV in OIR rats.

FIG. 9 shows retinal angiographs of OIR rats with a single intravitreal injection of thalidomide and Compounds. In FIG. 9A, OIR rats received an intravitreal injection of 5 μl of each compound (0.8 mM in BN rat serum) per eye into the right eye and the same volume of the vehicle into the left eye. Fluorescein retinal angiography was performed at P16. Angiographs are representatives of 3 rats per group. It is to be noted that Compound 4-injected rats have reduced NV, compared to the control. thalidomide, Compound 1, and Compound 2 did not reduce the NV at the dose used. In FIG. 9B, compared with vehicle-treated rat, the examination of the section showed that pre-retinal NV was decreased in eye treated with Compound 4.

### Example 11

### Rat Strain Difference in Vascular Leakage in the Retinas of OIR and STZ-Induced Diabetic Rats

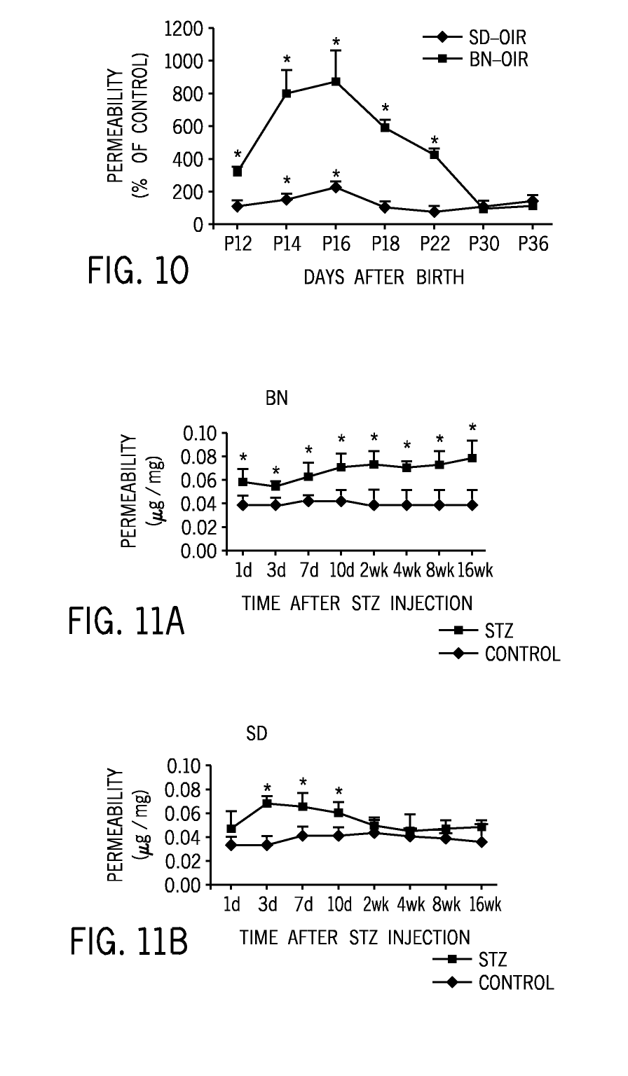

A model was established for sustained retinal vascular leakage for testing the long-term effect of new drugs. The time courses of retinal vascular permeability were defined in both the OIR and STZ-diabetic models in Sprague Dawley and BN rats. OIR was induced by exposing neonatal rats to hyperoxia (75% O₂) from P7 to P12. Diabetes was induced in adult BN rats by STZ injection. Retinal vascular permeability was measured using the Evans Blue-albumin method. In OIR-BN rats, the permeability started to increase at P12, reaching its peak at P16 with an 8.7-fold increase over the level in age-matched normal rats (P=7.5E-06). Between P18 and P22, the permeability slowly declined, reaching normal levels after P30 (FIG. 10). In OIR-SD rats, the permeability started to increase later (P14). The peak value was lower than that in BN rats (2.2-fold) and permeability declined to the normal level by P18 (FIG. 10). These observations correlated with different retina VEGF levels in the two strains. In STZ-BN rats, hyper-permeability occurred 24 h after the STZ injection (1.4-fold; P=0.0292) and reached a plateau at 2 wks (1.8-fold, P=0.0074). The hyper-permeability lasted at least 16 wks after the induction of diabetes. In STZ-SD rats, the permeability started to increase 3 days after the STZ-injection (1.3-fold; P=0.0271), reached its peak at 1 wk (1.5-fold; P=0.004) and declined to the control level by 2 wks (FIG. 11). These results suggest that in both OIR and STZ-diabetes, vascular leakage is significantly higher and lasts longer in BN than in SD rats. Therefore, all of the studies in this project involving rat models used BN rats. These results also suggest that the OIR model is good for short term effect while the STZ-diabetes model is suitable for evaluating long-term effect of Compound 4 on retinal vascular leakage as proposed in this Phase II project.

FIG. 10 shows the rat strain difference in vascular permeability in the OIR model. BN and SD rats were treated with hyperoxia and vascular permeability in the retina was measured, normalized by total protein concentration and expressed as percentages of that of respective age-matched normal control (mean±SD, n=4). Values significantly higher than the control are indicated by *.

FIG. 11 shows the strain difference in vascular permeability in STZ-diabetic model. Diabetes was induced in BN and SD rats and permeability in the retina was measured at different time points as indicated. Permeability was normalized by total protein concentrations and expressed as μg of Evans blue per mg of proteins (mean±SD, n=4). Values significantly higher than the age-matched normal control are indicated by *.

### Example 12

### BN Rats were Found to have Higher VEGF Levels in the Retina than SD Rats in Response to Ischemia

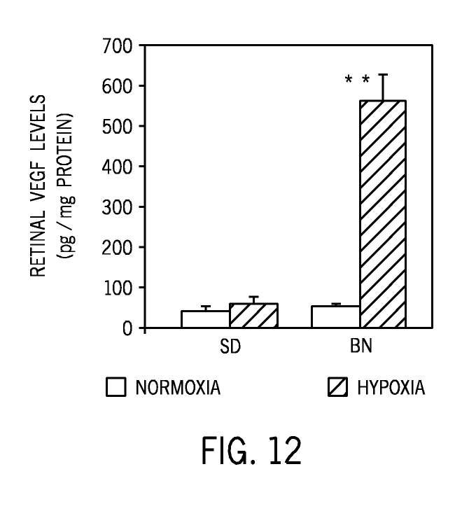

To determine if the more severe retina NV in BN rats are correlated with their retinal VEGF over-production in the OIR model, VEGF levels were quantified using a rat VEGF ELISA kit (R&D systems, Inc) and normalized by total retinal protein concentrations. The results showed that the basal level of retinal VEGF were similar in normal BN and SD rats. In OIR-SD rats, retinal VEGF levels had no significant difference compared with those in normal control SD rats (FIG. 12). However, retinal VEGF levels in OIR-BN rats were about 10-folds higher than those in normal control BN and OIR SD rats (P<0.001, n=4) (FIG. 12).

FIG. 12 shows VEGF levels in OIR BN and SD rats. The retinal VEGF levels were measured by ELISA, normalized by retinal protein and expressed as pg/mg protein (mean±SD, n=4). Value significantly higher than the age-matched normal control are indicated by * (P<0.001).

### Example 13

### BN Rats were Found to have Higher VEGF Induction in the Retina than SD Rats in Response to STZ-Induced Diabetes

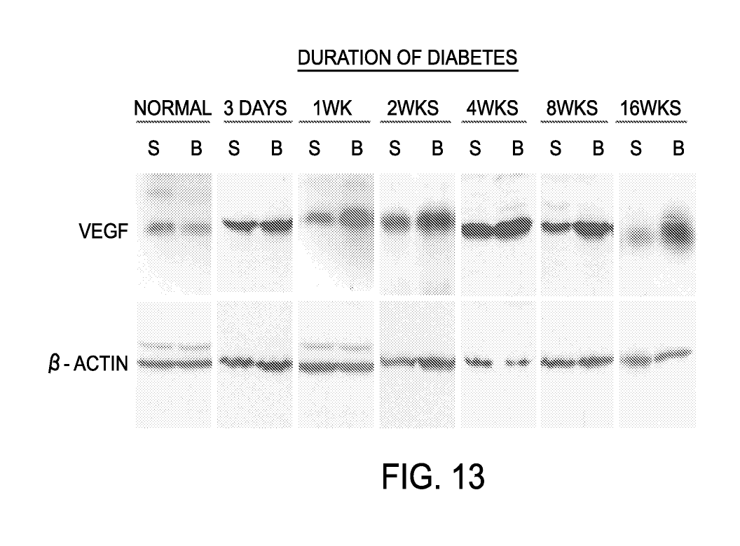

Studies have shown that BN rats with STZ-induced diabetes develop more severe retinal vascular leakage than STZ-diabetic SD rats with similar hyperglycemia and duration. To determine if the retinal VEGF expression is up-regulated more significantly in BN than in SD rats by diabetes, retinal VEGF levels were measured and semi-quantified by Western blot analysis in BN and SD rats with STZ-induced diabetes and compared to respective age-matched non-diabetic controls at different time points after the onset of diabetes. The results showed that the basal level of retinal VEGF expression was similar in normal adult BN and SD rats (FIG. 11). Following the induction of diabetes by STZ, however, the retinal VEGF levels in diabetic BN rats were higher than those in diabetic SD rats during the time period of 3 days to 16 weeks of diabetes (FIG. 13).

These observations suggest that the retinas of BN rats with OIR or STZ-diabetes are suitable in vivo models for investigating the mechanism of Compound 4, i.e, its effect on VEGF over-expression.

FIG. 13 shows retinal VEGF levels in BN and SD rats with STZ-diabetes. The retinas were dissected from diabetic BN and SD rats at 3 days, and 1, 2, 4, 8 and 16 weeks following the STZ injection. The same amounts of soluble proteins were blotted with an antibody specific to VEGF. The same filter was stripped and re-blotted with anti-β-actin antibody to normalize VEGF levels. The results are from pooled retinas of animals at each point.

### Example 14

### Compound 4 was not Found to Show any Detectable Ocular Toxicity in Rats

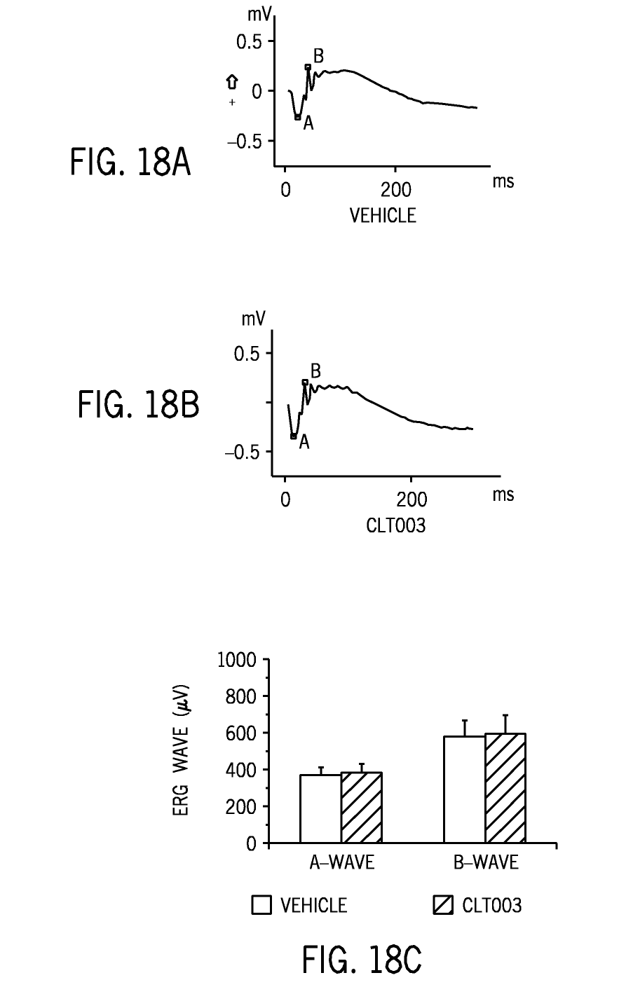

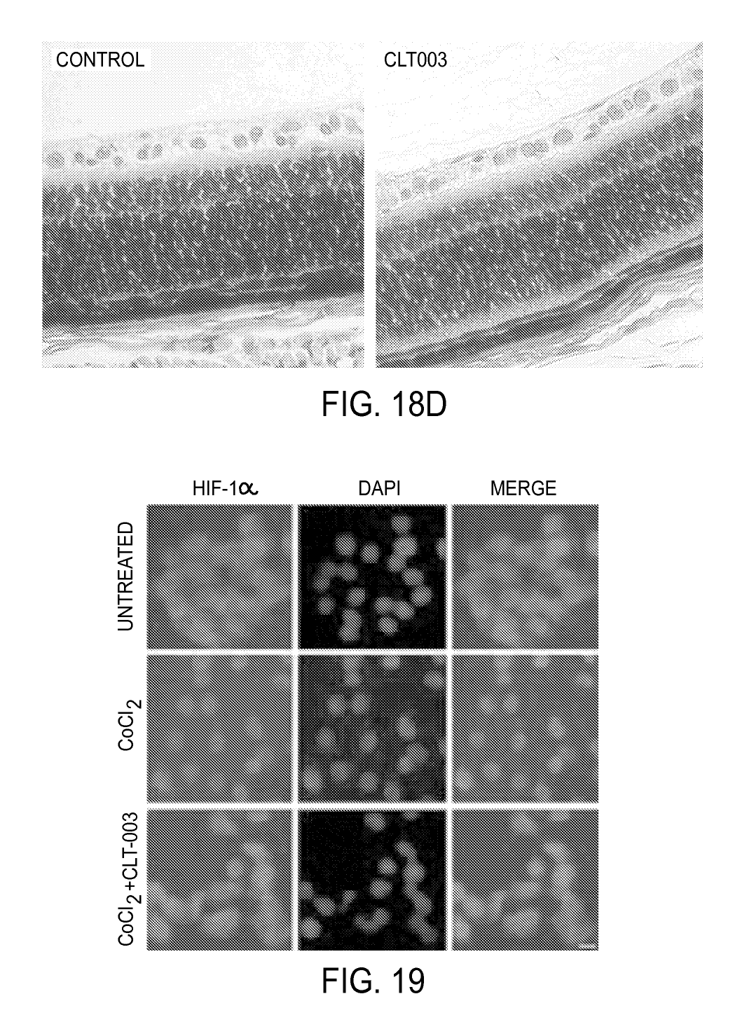

To test the potential ocular toxicities of Compound 4, normal rats at age of 8 weeks received an intravitreal injection of a high dose of Compound 4, 2 μg/eye (5 μl/eye of 0.4 mg/ml in BN rat serum) or an equal amount of BN rat serum as the vehicle control. Prior to study initiation, and after weeks 1, 2, 3, and 4 following the injection, visual function was evaluated by ERG recording. ERG recording showed no detectable change in the a-wave and b-wave amplitudes in Compound 4-injected rats compared to vehicle-injected eyes (FIG. 15 and FIG. 18A-C).

Possible toxicities of CLT-033 were also examined using pathohistological examination 4 weeks after the drug administration. Retinal cross sections stained with H&E were examined under a light microscope. No apparent morphological change or immunoresponse was found in the retinas treated with 2 μg/eye Compound 4 (5 μl/eye of 0.4 mg/ml), compared with the contralateral retina treated with the vehicle (FIG. 18D).

FIG. 15 is a table comparing the effect of Compound 4 on the A wave and b wave of eyes in rats.

FIG. 18 shows the functional and morphological analysis of the retina treated by Compound 4 in rats. Eight weeks old BN rats were received an intravitreal injection of Compound 4 (2.0 μg/eye, 5 μl/eye 0.4 mg/ml in BN rat serum) or equal amount of BN rat serum respectively (n=6). ERG was performed prior to study initiation and 1, 2, 3 and 4 weeks after the injection. Data shown no dramatic change in the a-wave and b-wave amplitudes in Compound 4-injected rats compared to vehicle-injected rats (FIG. 18A-18C). The animals were sacrificed 4 weeks after injection. The eye sections were observed under microscope with HE staining. Pathological observation showed that no detectable morphological change was found in the retinas of rats treated by Compound 4 and control (FIG. 18D).

### Example 15

### Compound 4 Attenuates HIF-1α Activation

According to embodiments and as illustrated by the experimental data in FIG. 19, the effect of Compound 4 on HIF-1α activation is shown. Human retinal pigment epithelial cells (ARPE-19) were incubated with 400 μM CoCl₂ for 16 h in the absence or presence of 0.5, 1, and 2 μM Compound 4. The nuclear translocation of HIF-1α was detected by immunolabeling. HIF-1α was stained green and nuclei were counterstained with DAPI (blue). Bar: 10 μm.

### Example 16

### Compound 4 Reduces the Expression of VEGF and ICAM-1 in Retinal Pigment Epithelial Cells

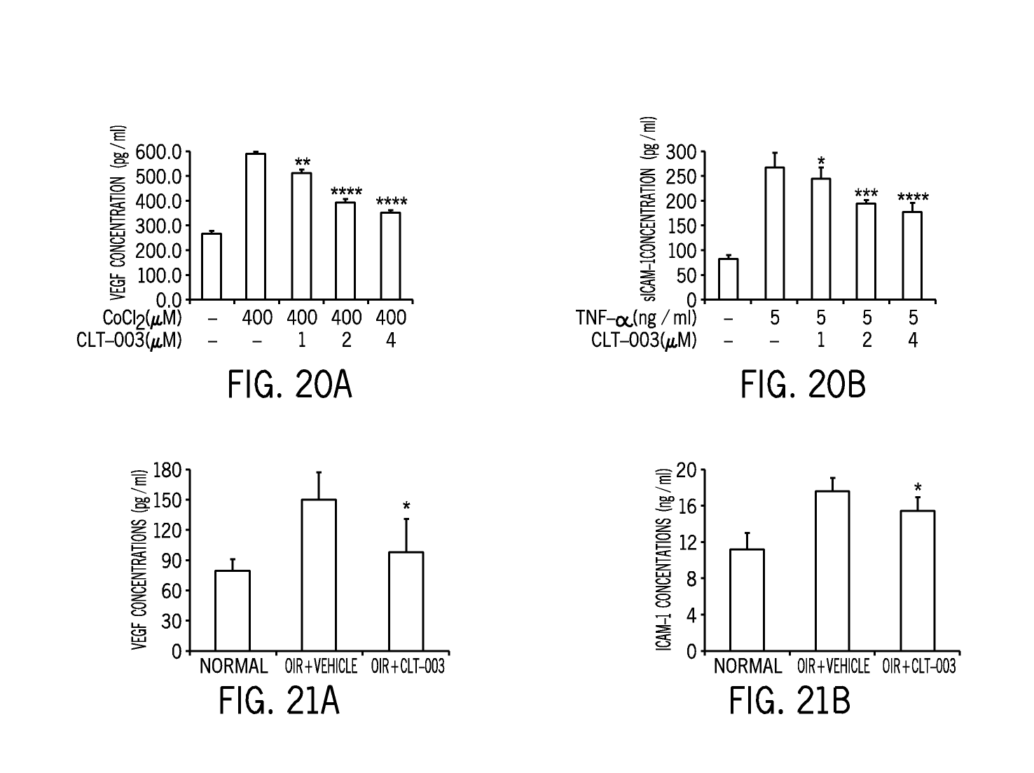

According to embodiments and as illustrated by the experimental data in FIG. 20, the effect of Compound 4 on the expression of VEGF and soluble ICAM-1 in ARPE-19 cells is shown. ARPE-19 cells were treated with CoCl₂, CoCl₂ plus Compound 4, TNF-α, and TNF-α plus Compound 4, respectively. Cell-free conditioned medium was collected 18 h after treatment and the levels of VEGF and soluble ICAM-1 were measured by ELISA. FIG. 20A shows the effect on VEGF levels; FIG. 20B shows the effect on soluble ICAM-1 levels. *p>0.05, **p<0.05, ***p<0.01, ****p<0.001, n=3.

### Example 17

### Compound 4 Down-Regulates the Expression of VEGF and ICAM-1 in the Retina of OIR Rats

According to embodiments and as illustrated by the experimental data in FIG. 21, the effect of Compound 4 on the expression of VEGF and ICAM-1 in the retina of OIR rats is shown. To induce OIR, BN rats at postnatal day 7 (P7) were exposed to hyperoxia (75% O₂) for 5 days (P7-P12) and then returned to normoxia. OIR rats received an intravitreal injection of Compound 4 and vehicle at P14. Two days after injections, VEGF and ICAM-1 levels in the retina from normal rats, vehicle treated OIR rats, and Compound 4-treated OIR rats were measured by ELISA. FIG. 21A shows the effect of Compound 4 on VEGF expression; FIG. 21B shows the effect of Compound 4 on ICAM-1 expression. * p<0.01, n=5.

### Example 18

### A Single Intravitreal Injection of Compound 4 Inhibits Retinal Vascular Leakage in OIR and STZ-Diabetes Rats

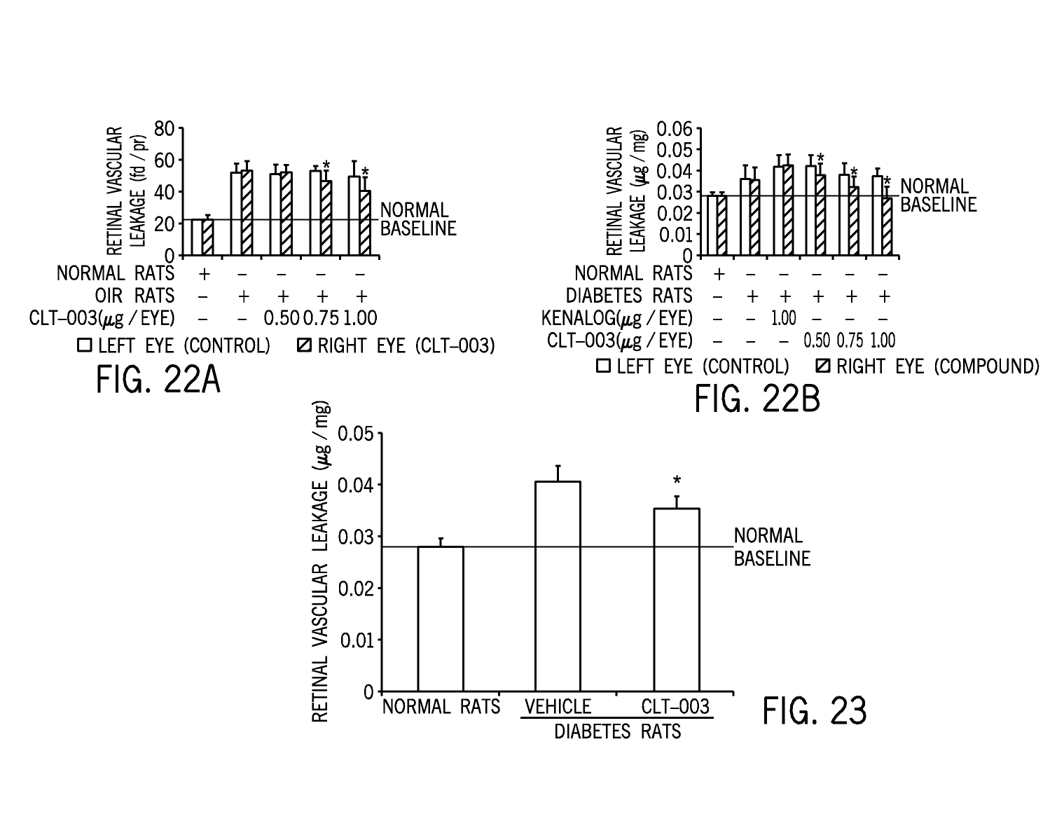

According to embodiments and as illustrated by the experimental data in FIG. 23, the effect of Compound 4 retinal vascular leakage in OIR and STZ-diabetic rats is shown. In FIG. 22A, OIR rats received an intravitreal injection of Compound 4 in right eye and vehicle in left eye for control at P14. Vascular leakage was measured using the FITC-albumin leakage method at P16. Compound 4 suppressed retinal vascular leakage at 0.75 and 1 μg/eye. In FIG. 22B, BN rats were induced by an intraperitoneal injection of STZ (50 mg/kg). At 2 weeks following the onset of diabetes, the diabetes rats received an intravitreal injection of Compound 4, triamcinolone acetonide (Kenalog) in right eye, and vehicle in the left eye for control. Two days following injections, retinal vascular leakage was quantified using the Evans Blue extravasation method. All doses of Compound 4 tested (0.5, 0.75, and 1 μg) produced a significant reduction in retinal vascular permeability. Most impressively, 1 μg of Compound 4 restored retinal vascular permeability to the baseline levels observed in normal rats. At en equal dose, Kenalog had no therapeutic effect. *p<0.05, n=5.

### Example 19

### Oral Administration of Compound 4 Suppresses Retinal Vascular Leakage in STZ-Diabetes Rats

According to embodiments and as illustrated by the experimental data in FIG. 23, the effect of Compound 4 on retinal vascular leakage in STZ-diabetes rats after oral administration is shown. One week after the induction of diabetes by STZ, diabetic rats received Compound 4 (once daily, 200 mg/kg/day) via gavage for 7 day; diabetic rats received the equal volume of the vehicle (corn oil) as a control. Retinal vascular permeability was measured using the Evans Blue extravasation method. Compound 4-treated rats were compared to control. *p<0.05, n=10.

### Example 20

### Encapsulation of Compound 4 Nanoparticles

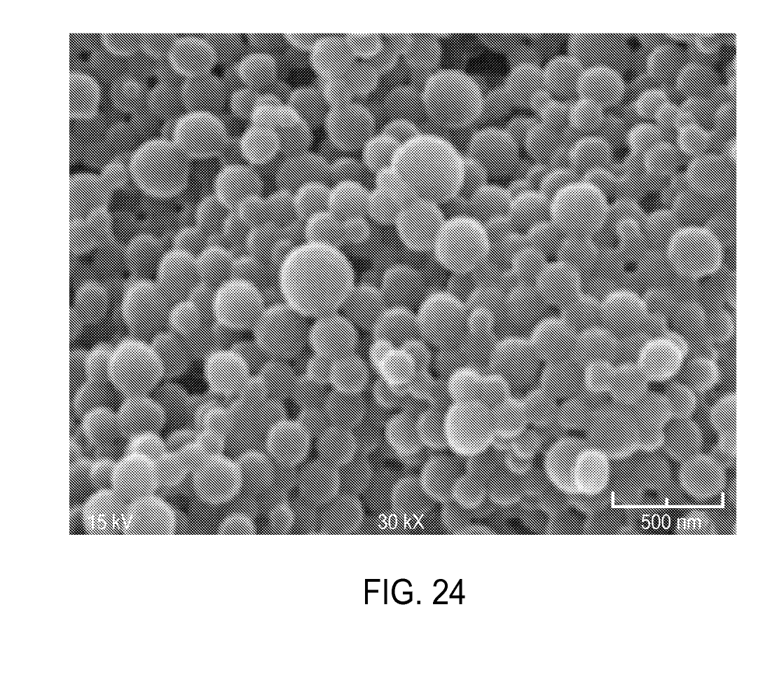

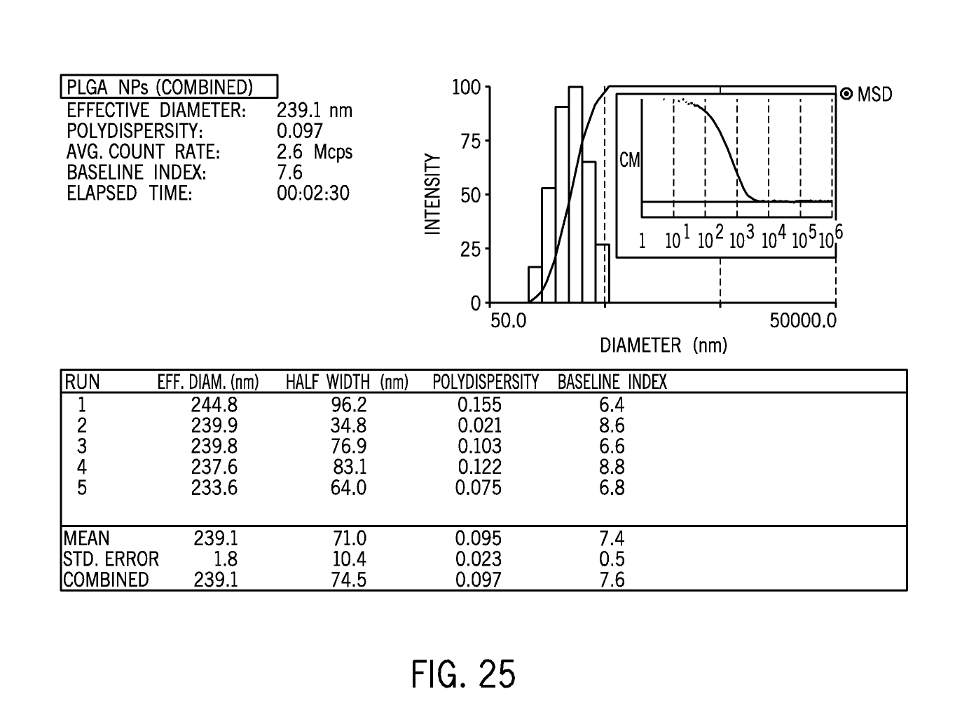

According to embodiments and as illustrated in FIG. 24 a scanning electron micrograph of Compound 4 in PLGA nanoparticles is shown. Accordingly PLGA nanoparticles containing Compound 4 were formulated using a water-in-oil emulsion solvent evaporation method. PLGA (comprised of 50, 65, 75, 85 percent lactic acid) with a viscosity ranging from 0.15 to 1.2 dL/g were dissolved in an 80% dichloromethane/20% dimethyl formamide to make a 7% solution. Compound 4 was added in concentrations ranging from 0 to 22 mg/ml. The PLGA compound 4 solution was then added to a 1 to 5% polyvinyl acid solution in a 1:2 oil to water ratio. The combined solution was sonicated in an ice bath at 30% amplitude with a Sonics Ultrasonic Processor (model VCX-750, ½ inch probe) for 1 minute. The solution was stirred vigorously overnight to allow the oil to evaporate off. The nanoparticles are then centrifuged twice at 15,000 g and washed each time with 18 megaohm water. Finally the nanoparticles are resuspended in 18 megaohm water by probe sonicating for 1 min. The nanoparticles are transferred to 1.7 ml cryovials and frozen overnight at −80° C. and then lyophilized for 48 hours. The particles are smooth and spherical. Dynamic light scattering analysis demonstrated that the nanoparticles produced in the formulation scheme had an average hydrodynamic diameter of 239.1 nm with a polydispersity index of 0.97 (FIG. 25). The Compound 4 loading in the nanoparticles was found to be between 10 and 36% w/w.

### Example 21

### 7. Compound 4 Nanoparticles have a Sustained Efficacy on Retinal Vascular Leakage in STZ-Diabetes Rats

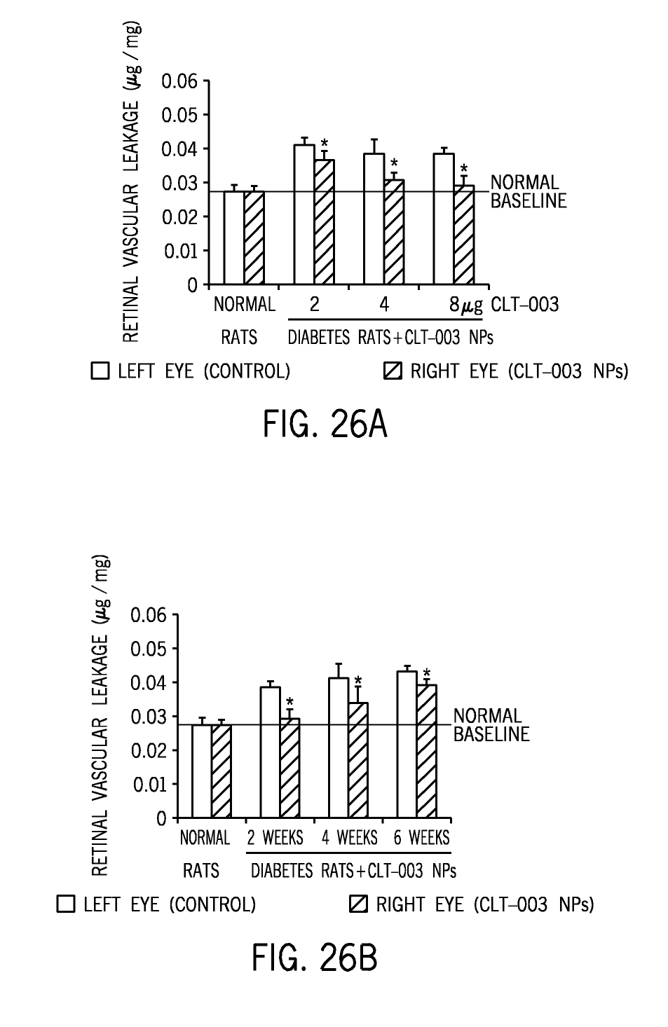

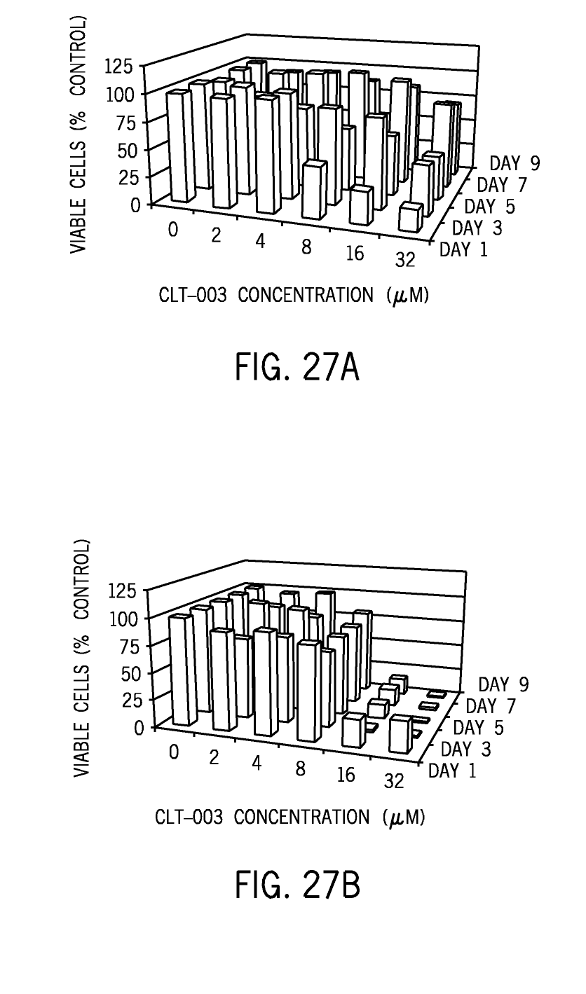

According to embodiments and as illustrated in FIG. 26, the effect of Compound 4 NPs on retinal vascular leakage in STZ-diabetes rats is shown. In FIG. 26A, two weeks after the induction of diabetes by STZ, diabetic rats received an intravitreal injection of Compound 4 NPs (13% w/w drug loading; 15, 30, and 60 μg/eye, 5 μl/eye of 3, 6 and 12 mg/ml in PBS, equivalent to 2, 4, 8 μg of Compound 4) in right eyes and the equal volume of PBS in the left eyes. Retinal vascular leakage was measured by Evans Blue extravasation method 2 weeks after the injection and normalized by the total protein concentration in the retina and the Evans blue concentration in the blood (mean±SD, n=5). Vascular permeability in non-diabetic rats was used as baseline of permeability. Compound 4 NPs inhibited retinal vascular leakage in a dose-dependent manner at the doses of 2, 4, 8 μg Compound 4/eye. In FIG. 26B shows STZ-diabetic rats that received an intravitreal injection of Compound 4-NPs (equivalent to 8 μg of Compound 4 for 2 and 4 weeks, 64 μg of Compound 4 for 6 weeks). Retinal vascular leakage was measured by Evans Blue extravasation method 2, 4, and 6 weeks after injections. Each of the experimental group was compared with the vehicle control. Compound 4 NPs had long-term inhibitory efficacy on retinal vascular leakage for 6 weeks. * p<0.05, n=5.

### Example 22

### Inhibitory Activity of Nanoparticles and Unincorporated Compound 4 on BRCEC Growth

To test the inhibitory effect of Compound 4 encapsulated in nanoparticles (NP) on endothelial cell growth, BRCECs were seeded in triplicate in gelatin-coated 24-well plates 24 hours prior to the experiments. Cells were treated with or without the control NPs and Compound 4 NPs (13% w/w drug loading) at the concentrations of 5, 10, 20, 40, and 80 μg/ml, as well as unincorporated Compound 4 at the concentrations of 2, 4, 8, 16 and 32 μM. Each treatment of Compound 4 NPs and unincorporated Compound 4 contained relatively equal amounts of Compound 4 in the well plates. Four hours after treatment, the medium was replaced by fresh medium without the NPs or Compound 4. Viable cells were quantified using the MTT assay at 1, 3, 5, 7 and 9 days after treatment. Compound 4 NPs showed significant inhibition of cell proliferation 1 day after treatment at concentrations of 40 and 80 μg/ml (16 and 32 μM of Compound 4) (n=3, p<0.05). Cell growth was suppressed for the full 9 days of the experiment (FIG. 27B). Control NPs did not show any inhibitory effect at the same concentrations (data not shown). The unincorporated Compound 4 did significantly inhibit cell growth 1 day after treatment at the concentrations of 8, 16, and 32 μM (n=3, p<0.05), however, cell growth recovered 3 days after treatment (FIG. 27A). These results demonstrate that Compound 4 NP has a potent and sustained inhibitory effect on BRCEC growth.

BRCECs were treated with the control NPs, Compound 4 NPs (13% w/w drug loading) and unincorporated Compound 4 for 4 h. Each treatment of Compound 4 NPs and unincorporated Compound 4 contained relatively equal amounts of Compound 4 in the well plates. The compounds were removed by replacing of the medium with fresh growth medium. Viable cells were quantified using MTT assay at the time points as indicated, as illustrated in FIG. 27A. The unincorporated Compound 4 did significantly inhibit cell growth 1 day after treatment at the concentrations of 8, 16, and 32 μM (n=3, p<0.05), but the inhibitory effect disappeared after 3 days as shown in FIG. 27B. Compound 4-NP (40 and 80 μg/ml, equivalent to 16 and 32 μM of Compound 4) showed significant and sustained inhibition of cell growth at all the time points analyzed (n=3, p<0.05).

### Example 23

### Overview of Synthesis of Compound 4

Commercially available 4-nitrophthalic anhydride (5) was coupled with 2,6-diisopropyl aniline (6) in acetic acid to afford (2,6-diisopropylphenyl)-5-nitro-1H-isoindole-1,3-dione (7) in 91% yield. The nitro group in intermediate (7) underwent a reduction using a transfer hydrogenation reaction to afford 99.2% pure of the desired API (Compound 4) in 91% yield, as shown in Scheme 1.

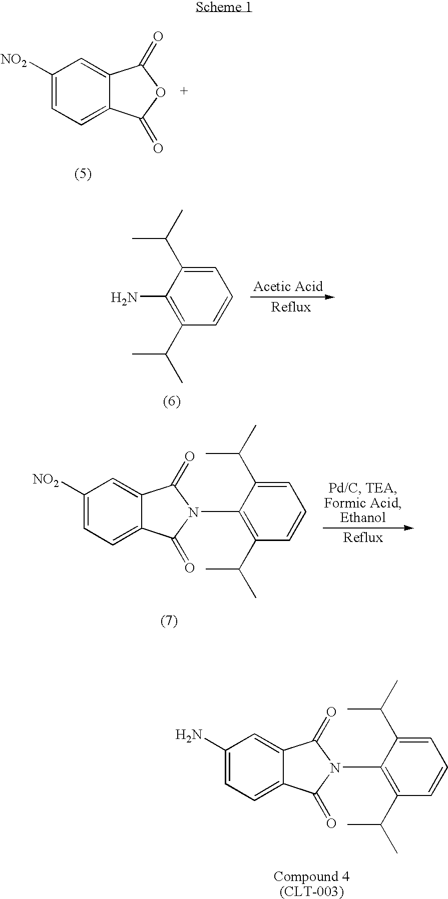

The coupling step to synthesize (2,6-diisopropylphenyl)-5-nitro-1H-isoindole-1,3-dione has been optimized resulting in a 91% yield with the solids having a purity of >99% (by HPLC (a/a %)). These results have remained consistent even upon scale-up. A recrystallization method has been developed to purify this intermediate, if needed. It has been proven that this purification method successfully removes the impurity formed when 3-nitrophthalic anhydride is present in the 4-nitrophthalic anhydride starting material. Based on these results, it is unnecessary to develop a purification of the 4-nitrophthalic anhydride prior to use.

Synthesis of Compound 4 was successfully carried out via catalytic hydrogenation and via transfer hydrogenation. Catalytic hydrogenation required a longer reaction time (24 h) under 45-50 psi. This method requires specialized equipment. It has been successfully demonstrated that the transfer hydrogenation reaction in ethanol (200 proof) is complete in 3 h in the presence of 10% (w/w) catalyst (with heat). Scale-up of this reaction gave 99.2% pure material in 91% yield. Preliminary work was demonstrated that drying at an elevated temperature (70° C.) for prolonged periods of time (89 h) does not cause decomposition and is successful in removing residual solvents.

A recrystallization of Compound 4 from ethanol/n-heptane (1:2) has been successful in producing 100% pure material with a 75% recovery.

### Example 24

### Synthesis of Compound 7 from Compounds 5 and 6

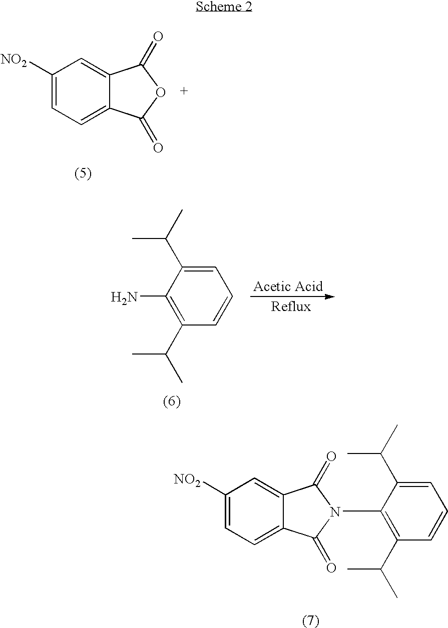

According to an embodiment, and as shown in Scheme 2,2,6-diisopropyl aniline (2.34 mL, 12.4 mmol, 1.2 eq) was added to a slurry of 4-nitrophthalic anhydride (2.0 g, 10.4 mmol) in acetic acid (15 mL). The reaction mixture was heated to reflux (resulting in a solution). After 12 h, the reaction was allowed to cool slowly to ambient temperature. The slurry was concentrated to a residue. Methanol (2×50 mL) was added to assist in the removal of acetic acid. The residue was crystallized from an ethyl acetate/petroleum ether mixture (1:2, 60 mL). The solids were isolated by filtration, rinsed with pet. ether (3-4 mL), and air-dried to afford white solids (2.43 g, 66% yield). ‘H NMR confirmed the structure. m.p. 168-169° C. (lit. 161-162° C.).

According to an embodiment, and as shown in Scheme 2,2,6-diisopropyl aniline (2.34 mL, 12.4 mmol, 1.2 eq) was added to a slurry of 4-nitrophthalic anhydride (2.0 g, 10.4 mmol) in acetic acid (15 mL). The reaction mixture was heated to reflux (resulting in a solution). After 12 h, the reaction was allowed to cool slowly to ambient temperature. The solids were isolated by filtration and rinsed with water (3 mL). The solids were recrystallized from ethyl acetate/heptane (1:2, 60 mL) to afford 2.23 g of a white powder (61% yield). The filtrate was concentrated to a residue. Methanol (2×25 mL) was added after the initial concentration to assist in the removal of acetic acid. During the second addition of methanol, the solids present were isolated by filtration and recrystallized from ethyl acetate/heptane (1:2, 3 mL) to afford 164 mg of white solids.

According to an embodiment, and as shown in Scheme 2,2,6-Diisopropyl aniline (2.34 mL, 12.4 mmol, 1.2 eq) was added to a slurry of 4-nitrophthalic anhydride (2.0 g, 10.4 mmol) in ethanol (200 proof, 40 mL). The reaction mixture was heated to reflux (resulting in a solution). After 12 h, the reaction was allowed to cool slowly to ambient temperature. By TLC it appeared the reaction was only 50% complete. The solution was re-heated to reflux for an additional 3 h. No significant change was noted. The reaction solution was allowed to cool to ambient temperature. Crystallization of the intermediate resulted in orange solids (0.93 g, 25% yield).

According to an embodiment, and as shown in Scheme 2,2,6-Diisopropyl aniline (9.4 mL, 49.72 mmol, 1.2 eq) was added to a slurry of 4-nitrophthalic anhydride (tech grade (90% pure), 8.0 g, 41.43 mmol) in acetic acid (60 mL). The reaction mixture was heated to reflux (resulting in a solution). After 12 h, the reaction was allowed to cool slowly to ambient temperature. The reaction mixture was concentrated to ca. half of the volume. The solids were isolated by filtration and rinsed with methanol (8 mL). The off-white solids were air-dried to afford 13.2 g (90% yield, 99.7% pure by HPLC (a/a %)). A sample of this intermediate (3.0 g) was recrystallized from a mixture of ethyl acetate/heptane (1:2, 63 mL). The white solids were isolated by filtration to afford 2.17 g of purified intermediate (72% recovery, 99.9% pure by HPLC (a/a %)).

According to an embodiment, and as shown in Scheme 2, 2,6-Diisopropyl aniline (16.5 g, 93.2 mmol, 1.2 eq) was added to a slurry of 4-nitrophthalic anhydride (15.0 g, 77.7 mmol) in acetic acid (113 mL). The reaction mixture was heated to reflux (resulting in a solution). After 12 h, the reaction was allowed to cool slowly to ambient temperature. The reaction mixture was concentrated to ca. half of the volume. The solids were isolated by filtration and rinsed with methanol (15 mL). The off-white solids were dried under vacuum at 35° C. to achieve constant weight. Afforded 24.7 g (90% yield, 99.7% pure by HPLC (a/a %)). ¹1-NMR confirms the structure.

### Example 25

### Synthesis of Compound 4 from Compound 7—Catalytic Hydrogenation

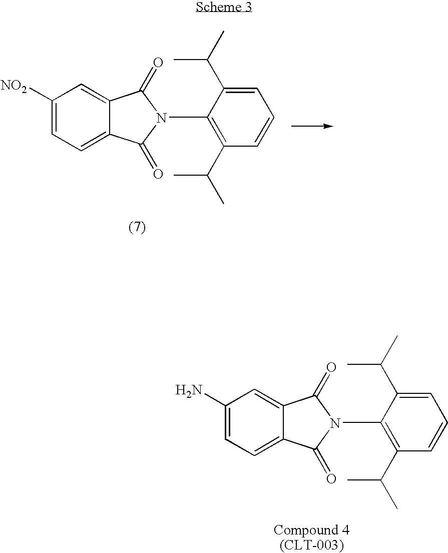

According to an embodiment, and as shown in Scheme 3, a slurry of (2,6-diisopropylphenyl)-5-nitro-1H-isoindole-1,3-dione (compound 7, 1.0 g, 2.84 mmol) and 10% Pd/C (10 mg, 1% (w/w)) in ethyl acetate (20 mL) was hydrogenated at 8-9 psi. The reaction progress was monitored by TLC. At 2 h, additional catalyst (40 mg, 5% total (w/w)) was added. Hydrogenation was continued overnight. TLC (silica gel, λ=254 nm, Heptane: Ethyl acetate (60:40)) showed a mixture of two spots at the same R_{f} but with different UV fluorescence. The reaction mixture was filtered through a Celite bed. The filter pad was rinsed with ethanol (200 proof, 2 mL). The filtrate was concentrated to a residue. The residue was crystallized from ethanol (7 mL) to afford 275 mg of yellow solids (30% yield). ¹H NMR and HPLC both confirmed a mixture of two compounds.

According to an embodiment, and as shown in Scheme 3, a slurry of compound 7 (0.5 g) and 10% Pd/C (25 mg, 5% (w/w)) in ethyl acetate (10 mL) was hydrogenated at 8-9 psi. After 2 h, the reaction mixture was filtered through a Celite bed. The filter pad was rinsed with ethyl acetate (3 mL). The filtrate was concentrated to a residue. ¹H NMR showed a mixture of two compounds (in a ca. 1:1 ratio). This residue was hydrogenated further (45 psi) in the presence of fresh catalyst (too mg) in ethyl acetate (20 mL). After hydrogenating overnight and isolation of a residue, ¹H NMR showed a significant reduction in the second set of peaks (in favor of a conversion to product).

According to an embodiment, and as shown in Scheme 3, a slurry of compound 7 (1.0 g) and 10% Pd/C (100 mg, 10% (w/w)) in ethyl acetate (20 mL) was hydrogenated at 45 psi for 23 h. The reaction mixture was filtered through a Celite bed. The filter pad was rinsed with ethyl acetate (6 mL). The filtrate was split into two equal portions. The first portion was concentrated to a residue. The second portion was treated with 12 N HCl and the solution was concentrated to a residue. The two sets of solids were compared by ¹H NMR. ¹H NMR confirmed the formation of the desired API and the HCl salt, respectively.

### Example 25

### Synthesis of Compound 4 from Compound 7—Transfer Hydrogenation

According to an embodiment, and as shown in Scheme 3, a mixture of compound 7 (1.0 g, 2.84 mmol) and 10% Pd/C (0.5 g, 5% (w/w)) in ethanol (200 proof, 16 mL) was treated with triethylamine (1.7 mL, 4.40 eq) and formic acid (0.55 g, 4.22 eq). The slurry was heated to reflux and the reaction progress was monitored by TLC. At 2 h, the reaction mixture was filtered through a Celite bed. The filter pad was rinsed with ethanol (2×1.5 mL). The filtrate was stirred at ambient temperature for 2 h. The solids were isolated by filtration to afford 0.49 g of fluorescent yellow solids (53% yield, 97.3% pure by HPLC (a/a %)). ¹H NMR confirmed the structure. A second crop was obtained from the filtrate (0.11 g, (comparable purity to the first crop)).

According to an embodiment, and as shown in Scheme 3, a mixture of compound 7 (0.5 g, 1.42 mmol) and 10% Pd/C (0.05 g, 10% (w/w)) in ethanol (200 proof, 8 mL) was treated with triethylamine (0.9 mL, 4.40 eq) and formic acid (0.2 mL, 4.22 eq). The reaction was monitored by TLC, while the reaction was kept at ambient temperature (via a cool water bath). At 40 min, the reaction mixture was filtered through a Celite bed. The filter pad was rinsed with ethanol (3×1 mL). Crystallization of the product from the filtrate by the addition of a co-solvent and cooling was unsuccessful. Therefore, the filtrate was concentrated to a residue. The residue was crystallized from an ethanol/heptane mixture (1:2, 6 mL). The solids were isolated by filtration to afford 0.26 g of yellow solids (56% yield, 99.2% pure by HPLC (a/a %)).

According to an embodiment, and as shown in Scheme 3, a mixture of compound 7 (to g, 2.84 mmol) and 10% Pd/C (0.1 g, 10% (w/w)) in ethanol (200 proof, 16 mL) was treated with triethylamine (1.8 mL, 4.40 eq) and formic acid (0.5 mL, 4.22 eq). The reaction mixture was heated to reflux and monitored by HPLC. At 3 h, the reaction mixture was filtered through a Celite bed. The filter pad was rinsed with ethanol (200 proof, 3×2 mL). The filtrate was stirred at ambient temperature overnight. The following day, the slurry was cooled and stirred at 0° C. for 1 h. The solids were isolated by filtration and dried at 35° C., under vacuum, to afford 488 mg of yellow solids (53% yield, 99.6% pure). A second crop of solids was isolated from the filtrate (138 mg, 99.4% pure by HPLC (a/a %)).

According to an embodiment, and as shown in Scheme 3, a mixture of compound 7 (1.0 g, 2.84 mmol) and 10% Pd/C (0.1 g, 10% (w/w)) in ethanol (200 proof, 16 mL) was treated with triethylamine (1.8 mL, 4.40 eq) and formic acid (0.5 mL, 4.22 eq). The reaction mixture was stirred at ambient temperature and monitored by HPLC. At 3 h, the reaction mixture was filtered through a Celite bed. The filter pad was rinsed with ethanol (200 proof, 6×2 mL). (Note: Additional washes were performed because it appeared some of the product had crystallized on the catalyst.) The filtrate was stirred at ambient temperature overnight. The following day, the slurry was cooled to 0° C. for 1 h. The solids were isolated by filtration and dried at 35° C., under vacuum, to afford 327 mg of yellow solids (36% yield, 99.7% pure). A second crop of solids was isolated from the filtrate (154 mg, 99.6% pure by HPLC (a/a %)).

According to an embodiment, and as shown in Scheme 3, a mixture of compound 7 (0.50 g, 1.42 mmol) and 10% Pd/C (0.05 g, 10% (w/w)) in ethyl acetate (8 mL) was treated with triethylamine (0.9 mL, 4.40 eq) and formic acid (0.2 mL, 4.22 eq). The reaction mixture was stirred at ambient temperature and monitored by HPLC. At 2.5 h, the reaction mixture was filtered through a Celite bed. The filter pad was rinsed with ethyl acetate (1×3 mL). The filtrate was concentrated to afford an oil (1.19 g). The oil was recrystallized from ethanol (200 proof, 4.5 mL) to afford 247 mg (54% yield, 98.8% pure by HPLC (a/a %)).

According to an embodiment, and as shown in Scheme 3, a mixture of compound 7 (5.0 g, 14.19 mmol) and 10% Pd/C (0.25 g, 5% (w/w)) in ethanol (200 proof, 80 mL) was treated with triethylamine (6.32 g, 4.40 eq). Formic acid (2.76 g, 4.22 eq) was added dropwise over 15 min, while maintaining the temperature <30° C. The slurry was heated to reflux and monitored by HPLC. The following day, additional catalyst (0.25 g in two portions) was added. When HPLC confirmed the reaction was complete, the reaction mixture was filtered through a Celite bed. The filter pad was rinsed with ethanol (200 proof, 2×12.5 mL). To this solution was added water (80 mL). Stirring was continued at ambient temperature for 2 h and then at 0° C. for 1 h. The solids were isolated by filtration and air-dried to afford orange-yellow colored solids as crude final product (4.1 g, 89% yield, 99.7% pure (a/a %)). ¹H NMR confirmed the structure.

According to an embodiment, crude compound 4 (2.0 g) was recrystallized from ethanol/water (1.6:1, 36 mL) to afford dark yellow solids (1.3 g, 65% recovery, 100% pure (a/a %)).

According to an embodiment, crude compound 4 (2.0 g) was recrystallized from ethanol/n-heptane (1:2, 66 mL) to afford bright yellow solids (1.5 g, 75% recovery, 100% pure (a/a %)).

### Example 27

### Large Scale Synthesis of Compound 4

According to embodiments, a 5 L, 3-neck round bottom flask, equipped appropriately and under nitrogen, was charged with 4-nitrophthalic anhydride (295.0 g, 1.53 mol), acetic acid (2.21 L, 7.5 mL/g), and 2,6-diisopropyl aniline (326.2 g, 1.84 mol, 1.2 eq). The slurry was heated to reflux for 12 h and then allowed to cool slowly to ambient temperature overnight. The reaction mixture was concentrated to ca. half of the volume (via distillation; removed ca. 1.33 kg). The slurry was cooled to ambient temperature and the solids were isolated by filtration and rinsed with methanol (3×100 mL). The solids were air-dried for 1-1.5 h and then dried further under vacuum, at 40° C., until constant weight was achieved. (2,6-diisopropylphenyl)-5-nitro-1H-isoindole-1,3-dione was produced as an off-white solid (488.0 g, 91% yield, 99.3% pure by HPLC (a/a %)). ¹H and ¹³C NMR confirmed the structure. m.p. 167-169° C. Elemental analysis: Theory: C (68.17); H (5.72); N (7.95); Found: C (68.08); H (5.77); N (7.86).

A 12 L, 3-neck round bottom flask, equipped appropriately and under nitrogen, was charged with a slurry of 10% palladium on carbon (24.5 g, 10% (w/w)) in ethanol (200 proof, 1 L). To this slurry was charged (2,6-diisopropylphenyl)-5-nitro-1H-isoindole-1,3-dione (245.0 g, 0.70 mol) followed by ethanol (200 proof, 2.92 L, 3.92 L total, 16 mL/g) and triethylamine (311.7 g, 3.08 mol, 4.40 eq). Formic acid (135.8 g, 2.95 mol, 4.22 eq) was added slowly over 25 min. The slurry was heated to reflux and the reaction progress was monitored by HPLC. At 3.5 h, the mixture was filtered through a Celite bed to remove the catalyst. The filter bed was rinsed with ethanol (200 proof, 4×175 mL). The filtrate was then clarified through Whatman filter paper to remove any particulates. Ethanol (200 proof, 2×150 mL) was used as a rinse. To the solution was added water (deionized, 3.92 L), over 45 min. The resulting slurry was stirred at ambient temperature overnight. The following day, the slurry was cooled to 0-10° C. for 2 h. The solids were isolated by filtration and rinsed with cold (0-10° C.) ethanol (200 proof; 2×200 mL). The solids were dried under vacuum, at 40° C., until constant weight was achieved. An in-process ¹H NMR showed an elevated amount of ethanol present. A small study was carried out to evaluate further drying at elevated temperature (70° C.). Based on the results (which showed no degradation of the final product), the solids were dried further under vacuum, at 70° C., until ¹H NMR confirmed the absence of ethanol. (2,6-diisopropylphenyl)-5-amino-1H-isoindole-1,3-dione, Compound 4, was afforded as a yellow solid (206.0 g, 91% yield, 99.2% purity by HPLC (ala %)). 1H and ¹³C NMR confirmed the structure. m.p. 259-261° C. Elemental analysis: Theory: C (74.51); H (6.88); N (8.69); Found: C (74.70); H (6.86); N (8.59).

While the method and agent have been described in terms of what are presently considered to be the most practical and preferred embodiments, it is to be understood that the disclosure need not be limited to the disclosed embodiments. It is intended to cover various modifications and similar arrangements included within the spirit and scope of the claims, the scope of which should be accorded the broadest interpretation so as to encompass all such modifications and similar structures. The present disclosure includes any and all embodiments of the following claims.

1. A method comprising:
administering to a subject having retinal edema a pharmaceutical composition having the chemical structure:

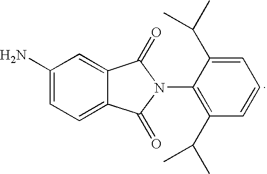

2. The method of claim 1, wherein the pharmaceutical composition is contained within a poly(lactic-co-glycolic acid) nanoparticle.

3. The method of claim 1, wherein the retinal edema is further characterized as macular edema.

4. The method of claim 3, wherein the retinal edema is further characterized as diabetic macular edema.

5. A method comprising:
providing a pharmaceutical composition to a patient experiencing retinal edema, the pharmaceutical composition comprising an agent having the chemical structure: 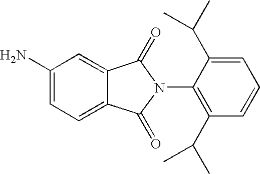
encapsulated in a poly(lactic-co-glycolic acid) nanoparticle.

6. The method of claim 5, wherein the retinal edema further characterized as macular edema.

7. The method of claim 6, wherein the macular edema is further characterized as diabetic macular edema.

8. A method comprising:
sustaining the efficacy of a pharmaceutical composition comprising a compound having the structure: 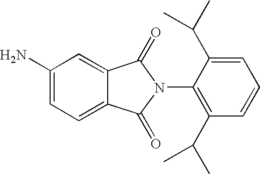
by encapsulating the compound in a poly(lactic-co-glycolic acid) nanoparticle;
wherein subsequent delivery of the encapsulated capsule causes the therapeutic effect of the compound for the treatment of diabetic macular edema to be extended.

9. The method of claim 8, wherein the retinal edema further characterized as macular edema.

10. The method of claim 9, wherein the macular edema is further characterized as diabetic macular edema.

11. A method comprising:
synthesizing a composition of the formula 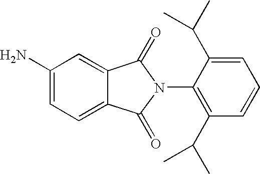
by coupling 4-nitrophthalic anhydride and 2,6-diisopropyl aniline by refluxing in acetic acid to produce (2,6-diisopropylphenyl)-5-nitro-1H-isoindole-1,3-dione; and
hydrogenating the (2,6-diisopropylphenyl)-5-nitro-4H-isoindole-1,3-dione by reacting the (2,6-diisopropylphenyl)-5-nitro-1H-isoindole-1,3-dione via a catalytic hydrogenation or transfer hydrogenation reaction.

12. The method of claim 11, wherein the catalytic hydrogenation comprises reacting the (2,6-diisopropylphenyl)-5-nitro-1H-isoindole-1,3-dione in ethyl acetate in the presence of Pd/C.

13. The method of claim 12, wherein the catalytic hydrogenation reaction is performed at a pressure above 5 psi.

14. The method of claim 11, wherein the transfer hydrogenation comprises reacting the (2,6-diisopropylphenyl)-5-nitro-1H-isoindole-1,3-dione in ethanol in the presence of Pd/C, triethylamine, and formic acid.

15. The method of claim 14, wherein the formic acid is added dropwise while the temperature of the reaction is maintained no greater than 30° C.
